(19) United States
(12) Patent Application Publication
(10) Pub. No.: US 2018/0125936 A1
(43) Pub. Date: May 10, 2018

(54) METHOD OF TREATING NEOPLASIAS

(71) Applicant: PARANTA BIOSCIENCES LIMITED, Melbourne (AU)

(72) Inventors: David N. WATKINS, Brighton (AU); Kieren D. MARINI, Mount Waverley (AU); David Morritz DE KRETSER, Surrey Hills (AU)

(73) Assignee: Paranta Biosciences Limited, Melbourne (AU)

(21) Appl. No.: 15/564,183

(22) PCT Filed: Mar. 23, 2016
(86) PCT No.: PCT/AU2016/050213

§ 371 Date: Oct. 3, 2017

(30) Foreign Application Priority Data
Apr. 7, 2015 (AU) 2015901244

(51) Int. Cl. A61K 38/18; A61K 31/255; A61K 31/4439; A61K 31/555; A61K 33/24; A61K 38/17; A61K 38/22; A61K 39/00; A61K 45/00; A61P 35/00

(52) U.S. Cl. CPC A61K 38/18; A61K 31/255; A61K 31/4439; A61K 31/555; A61K 33/24; A61K 38/1709; C12N 2310/127; A61K 38/22; A61K 39/0011; A61K 45/05; A61P 35/00; A61K 2300/00; C12N 2310/16; A61K 38/177

(57) ABSTRACT

The present invention relates generally to a method of treating a neoplastic condition. More particularly, the present invention is directed to a method of selectively sensitising neoplastic cells prior to chemotherapy. The method of the present invention is predicated on administering chemotherapy treatment subsequently to neoplastic cell sensitisation via the exposure of these cells to an activin type 1 B receptor (ACVR1B) antagonist. The present findings have now enabled the development of a new neoplastic treatment regime exhibiting both higher efficacy and reduced side effects for patients and, still further, a means of effectively treating chemoresistant neoplasms.

### FIELD OF THE INVENTION

The present invention relates generally to a method of treating a neoplastic condition. More particularly, the present invention is directed to a method of selectively sensitising neoplastic cells prior to chemotherapy. The method of the present invention is predicated on administering chemotherapy treatment subsequently to neoplastic cell sensitisation via the exposure of these cells to an activin type 1 B receptor (ACVR1B) antagonist. The present findings have now enabled the development of a new neoplastic treatment regime exhibiting both higher efficacy and reduced side effects for patients and, still further, a means of effectively treating chemoresistant neoplasms.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Malignant tumours, or cancers, grow in an uncontrolled manner, invade normal tissues, and often metastasize and grow at sites distant from the tissue of origin. In general, cancers are derived from one or only a few normal cells that have undergone a poorly understood process called malignant transformation. Cancers can arise from almost any tissue in the body. Those derived from epithelial cells, called carcinomas, are the most common kinds of cancers. Sarcomas are malignant tumours of mesenchymal tissues, arising from cells such as fibroblasts, muscle cells, and fat cells. Solid malignant tumours of lymphoid tissues are called lymphomas, and marrow and blood-borne malignant tumours of lymphocytes and other hematopoietic cells are called leukemias.

Cancer is one of the three leading causes of death in industrialised nations. As treatments for infectious diseases and the prevention of cardiovascular disease continues to improve, and the average life expectancy increases, cancer is likely to become the most common fatal disease in these countries. Therefore, successfully treating cancer requires that all the malignant cells be removed or destroyed without killing the patient. An ideal way to achieve this would be to induce an immune response against the tumour that would discriminate between the cells of the tumour and their normal cellular counterparts. However, immunological approaches to the treatment of cancer have been attempted for over a century with unsustainable results.

Accordingly, current methods of treating cancer continue to follow the long used protocol of surgical excision (if possible) followed by radiotherapy and/or chemotherapy, if necessary. The success rate of this rather crude form of treatment is extremely variable but generally decreases significantly as the tumour becomes more advanced and metastasises. Further, these treatments are associated with severe side effects including disfigurement and scarring from surgery (e.g. mastectomy or limb amputation), severe nausea and vomiting from chemotherapy, and most significantly, the damage to normal tissues such as the hair follicles, kidney, the peripheral nervous system, inner ear, gut and bone marrow which is induced as a result of the relatively non-specific targeting mechanism of the toxic drugs which form part of most cancer treatments.

Solid tumours cause the greatest number of deaths from cancer and mainly comprise tumours of the linings of the bronchial tree and the alimentary tract that are known as carcinomas. In fact, lung cancer is one of the most commonly fatal of human tumors. Many genetic alterations associated with development and progression of lung cancer have been reported, but the precise molecular mechanisms remain unclear (Sozzi, (2001) Eur J Cancer; 37 Suppl 7:S63-73). Small cell lung cancer (SCLC) is an aggressive, poorly differentiated neuroendocrine carcinoma with clinical, pathological and molecular characteristics distinct from those of non-small cell lung cancer (NSCLC). Clinically, SCLC is characterised by rapid growth, early metastatic spread and initial responsiveness to cytotoxic chemotherapy and ionizing radiation. The primary cause of SCLC is tobacco smoking, with well over 95% of patients being current or former smokers (Pesch et al. Int J Cancer 2012 131:1210-19).

Over one third of lung cancers are adenocarcinomas, a highly aggressive malignancy that arises in the distal airway compartment. Recent breakthroughs in targeted therapy directed at activating mutants of EGFR or ALK have highlighted the need for precise molecular classification of this disease. However, almost all patients treated with such therapies relapse, and are then treated with conventional chemotherapy. For the vast majority, patients with advanced disease whose tumours lack such targetable mutations, the prognosis remains grim. Therefore, a large unmet clinical need exists for improving the efficacy of conventional chemotherapy for the treatment of lung adenocarcinoma.

Solid tumours are not usually curable once they have spread or ‘metastasised’ throughout the body. The prognosis of metastatic solid tumours has improved only marginally in the last 50 years. The best chance for the cure of a solid tumour remains in the use of local treatments such as surgery and/or radiotherapy when the solid tumour is localised to its originating lining and has not spread either to the lymph nodes that drain the tumour elsewhere. Nonetheless, even at this early stage, and particularly if the tumour has spread to the draining lymph nodes, microscopic deposits of cancer known as micrometastases may have already spread throughout the body and will subsequently lead to the death of the patient. In this sense, cancer is a systemic disease that requires systemically administered treatments. Of the patients who receive surgery and/or radiotherapy as definitive local treatment for their primary tumour and who have micrometastases, a minor proportion may be cured or at least achieve a durable remission from cancer by the addition of adjuvant systemic treatments such as cytotoxic chemotherapy or hormones.

In this regard Cis-Diaminedichloroplatinum (CDDP) or cisplatin has been the cornerstone of chemotherapy for over 25 years. Cisplatin is a DNA reactive reagent widely used as a chemotherapeutic drug in the treatment of several kinds of human malignancies (Loehrer and Enihorn, 1984). The lesions that cisplatin forms with DNA are believed to be essential for the cytotoxic activity of the drug (Bruhn et al., 1993). Cisplatin binds to the N7 position of the imidazole ring of purines, predominantly guanine. The adducts of cis-DDP include intrastrand and interstrand 1,2-d(GpG), 1,2-d(ApG), 1,3-d(GpNpG) crosslinks (Eastman, 1983; Fichtinger-Shepman et al., 1985). The trans isomer of DDP, trans-diamminedichloroplatinum (II) (trans-DDP) is 20-fold less cytotoxic than cis-DDP (Pascoe and Roberts, 1974) and is ineffective against tumours. Trans-DDP forms similar adducts to those of cis-DDP with the exception that it cannot form 1,2-intrastand cross links (Pinto and Lippard, 1985) which represents greater than 90% of all adducts formed by cisplatin. As a consequence of the damage to DNA caused by both platinum compounds, DNA replication is blocked and additional cis-DDP induces a block in gene transcription.

Since cisplatin, and also carboplatin, appear to be most effective in cancers with defective DNA repair mechanisms, DNA is largely regarded as the molecular target of these agents. However, despite decades of use in the clinic, a complete understanding of how these drugs work is not clear. For example, only 1% of cisplatin that enters the cell binds to DNA, the remainder binds covalently to RNA and proteins. Furthermore, the degree to which these DNA-independent “off target effects” mediate cell death through mechanisms such as ER stress, death receptor activation or translational blockade, is highly concentration dependent, suggesting that platinum agents are complex therapeutic molecules with multiple targets and numerous potential resistance mechanisms. Further, the off target effects contribute to undesirable non-hematologic toxicity, including nausea, ototoxicity, nephrotoxicity and peripheral neuropathy.

More recently, the targeted therapy of cancer has aimed to improve the therapeutic ratio of cancer treatment by enhancing its specificity and/or precision of delivery to malignant tissues while minimising adverse consequences to normal non-malignant tissues. Two of the major classes of targeted therapy are (i) the small molecule inhibitors such as the tyrosine kinase inhibitors imatinib mesylate (Glivec®), gefitinib (Iressa®) and erlotinib (Tarceva®), and (ii) the monoclonal antibodies (mAb) such as rituximab (Mabthera®) and trastuzumab (Herceptin®).

In parallel to the development of targeted therapies, combining at least two conventional anti-cancer treatments such as chemotherapy and radiotherapy in novel ways has been another approach to the development of cancer therapeutics. By exploiting synergistic interactions between the different modalities of treatment, combined modality treatment seeks to improve treatment efficacy so that the therapeutic ratio for the combined treatment is superior to that for each of the individual treatments.

Combined modality treatment using external beam radiation and radiosensitising chemotherapeutic drugs such as 5-fluorouracil and cisplatin (chemoradiotherapy) has improved survival in a number of solid tumours such as those of head and neck, lung, oesophagus, stomach, pancreas and rectum because of both improved local tumour control and reduced rates of distant failure (T S Lawrence. Oncology (Huntington) 17, 23-28, 2003). Although radiosensitising drugs increase tumour response, they also increase toxicity to adjacent normal tissues, which is especially true of the potent new generation radiosensitisers, gemcitabine and docetaxel. However, decreasing the radiation volume allows cytotoxic doses of gemcitabine to be better tolerated clinically (Lawrence T S. Oncology (Huntington) 17, 23-28, 2003). Chemoradiotherapy may overcome mutually reinforcing resistance mechanisms, which may only manifest in vivo.

Radioimmunotherapy (RIT) is a systemic treatment that takes advantage of the specificity and avidity of the antigen-antibody interaction to deliver lethal doses of radiation to cells that bear the target antigen. Radio-isotopes that emit β-particles (e.g. ¹³¹Iodine, ⁹⁰Yttrium, ¹⁸⁸Rhenium, and ⁶⁷Copper) are usually used to label monoclonal antibodies (mAb) for therapeutic applications. The energy from β-radiation is released at relatively low intensity over distances measured in millimeters (Waldmann, Science 252: 1657-1662, 1991; Bender et al., Cancer Research 52: 121-126, 1992; O'Donoghue et al. Journal of Nuclear Medicine 36: 1902-1909, 1995; Griffiths et al. International Journal of Cancer 81: 985 992, 1999). Thus, high-energy β-emitters such as ⁹⁰Yttrium are useful for the treatment of larger and heterogeneous solid tumours (Liu et al. Bioconjugate Chemistry 12:7-34, 2001). Furthermore, the lower but biologically effective dose of radiation delivered by RIT had greater cytocidal effects than a larger dose of radiation conveyed as external beam radiotherapy (Dadachova et al. Proceedings of the National Academy of Sciences of the United States of America 101: 14865-14870, 2004). Nonetheless, the efficiency of RIT as a treatment for solid tumours may be hampered by the low penetration of antibody through the tissue barriers that surround the target antigen in the tumour, which will consequently extend circulatory half life of the antibody (Britz-Cunningham et al. Journal of Nuclear Medicine 44: 1945-1961, 2003). Furthermore, RIT is often impeded by the heterogeneity of the target antigen's expression within the tumour. Thus, although RIT affords molecular targeting of tumour cells, the major limitation of RIT remains the toxicity that may result from large doses of radiation that are delivered systemically in order to achieve sufficient targeting (Britz-Cunningham et al. 2003, supra; Christiansen et al. Molecular Cancer Therapy 3: 1493-1501, 2004). Altogether, a useful therapeutic index using RIT has proven difficult to achieve clinically (Sellers et al. Journal of Clinical Investigation 104: 1655 1661, 1999).

Tumour associated antigens, which would allow differential targeting of tumours while sparing normal cells, have also been the focus of cancer research. Although abundant ubiquitous antigens may provide a more concentrated and accessible target for RIT, studies adopting this have been extremely limited.

Aside from the severe problem of side effects, one of the other limitations of chemotherapy is the development of resistance to the drug. For example, small cell lung cancer initially responds to platinum treatment, however after subsequent rounds of platinum chemotherapy, cells will often develop chemoresistance. Initial response rates for adenocarcinoma remain below 20%, with only marginal improvements in overall survival, suggesting that some tumours are in fact innately chemoresistant. More than 147 potential cell autonomous mechanisms of platinum resistance have been proposed across at least a dozen tumour types. Passive mechanisms include decreased cellular uptake, decreased drug binding, down-regulation of cell death pathways and increased tolerance to DNA damage. Active mechanisms include increased efflux, accelerated detoxification, enhanced DNA repair, and upregulation of anti-apoptosis mechanisms. Clearly, this presents a very complex problem.

Accordingly, there is an urgent and ongoing need to develop improved therapies for treating cancers.

In work leading up to the present invention it has been unexpectedly determined that antagonising activin type 1 B receptor (ACVR1B), optionally together with antagonising TGF Beta Receptor 1 (TGFBR1) mediated signalling, in a neoplastic cell preferentially sensitises a neoplastic cell, but not non-neoplastic cells, to platin agents and or alkylating agents. Where a cell is chemonaive, this is extremely valuable since it not only enables a higher likelihood of cure but, further, may enable the use of either lower concentrations of chemotherapy or else a shortened period of treatment. In addition to their therapeutic efficacy, it has also been unexpectedly determined that specifically antagonising ACVR1B provides other very significant advantages, which were previously not attainable, such as reduced chemotoxicity of the bone marrow and kidneys, anti-cachectic functionality and a reduction in patient fatigue. Accordingly, aside from the potential therapeutic benefits, the reduction of the severity and extent of side effects in a patient is in itself highly desirable. Still further, even where resistance to an alkylating agent or platin agent either pre-exists or has developed, it has been determined that the downregulation of ACVR1B-mediated signalling can effectively reverse the resistant state of the cell and thereby restore the sensitivity of that cell to therapy, thereby avoiding the need to switch the patient to new, and possibly inferior, therapeutic treatment regimes.

In yet another aspect it has been unexpectedly determined that simultaneously antagonising the functionality of each of GDF11 and activin A particularly effectively sensitises a neoplastic cell, but not non-neoplastic cells, to platin agents and alkylating agents. The therapeutic outcomes which are achieved were where these two ligands are antagonised together are surprisingly better than other combinations of ligands which one may select for the ACVR1B signalling pathway. Although targeting other combinations of ligands or receptors does provide a useful therapeutic outcome, antagonising the combination of these two specific ligands has been determined to be particularly efficacious. In this regard, prior art methods of sensitisation, to date, have focussed on modulating an isolated target. The present inventors, however, have determined that a specific set of ligands, which operate across multiple intracellular signalling pathways, provide an unexpectedly efficacious therapeutic outcome if they are antagonised simultaneously.

The method of the present invention thereby provides a means of delivering chemotherapy based treatment in a manner that can substantially improve both the survival and the quality of life of a patient. This is achieved not only by increasing the sensitivity of neoplastic cells to treatment but, further, alleviating the level of kidney and bone marrow toxicity (and thereby its associated complications) which would otherwise be expected to occur. Accordingly, significantly improved outcomes and/or reduced side effects relative to those which would normally be expected in the context of a conventional treatment regime are now achievable.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The subject specification contains protein sequence information prepared using the programme PatentIn presented herein after the bibliography. Each protein sequence is identified in the sequence listing by the numeric indicator <210> followed by the sequence identifier (eg. <210>1, <210>2, etc). The length, type of sequence (PRT, etc) and source organism for each sequence is indicated by information provided in the numeric indicator fields <211>, <212>, <213>, respectively. Protein sequences referred to in the specification are identified by the indicator SEQ ID NO: followed by the sequence identifier (eg. SEQ ID NO:1, SEQ ID NO:2, etc). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing.

One aspect of the present invention is directed to a method for the treatment of a neoplastic condition in a subject, said method comprising:
(i) administering to said subject an antagonist which downregulates ACVR1B-mediated signalling for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

Another aspect of the present invention is directed to a method for the treatment of a neoplastic condition in a subject, said method comprising:
(i) administering to said subject an antagonist of the functionality of each of GDF11 and activin A for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

In yet another aspect there is provided a method for the treatment of a carcinoma in a subject, said method comprising:
(i) administering to said subject an antagonist which downregulates ACVR1B-mediated signalling for a time and under conditions sufficient for said antagonist to increase the sensitivity of said carcinoma to an agent which downregulates neoplastic cell growth, which agent is an alkylating agent or a platin agent; and
(ii) administering said agent to said subject for a time and under conditions sufficient to downregulate the neoplastic cell growth of said carcinoma.

In still another aspect there is provided a method for the treatment of a carcinoma in a subject, said method comprising:
(i) administering to said subject an antagonist of the functionality of each of GDF11 and activin A for a time and under conditions sufficient for said antagonist to increase the sensitivity of said carcinoma to an agent which downregulates neoplastic cell growth, which agent is an alkylating agent or a platin agent; and
(ii) administering said agent to said subject for a time and under conditions sufficient to downregulate the neoplastic cell growth of said carcinoma.

In yet still another aspect there is provided a method for the treatment of a carcinoma in a subject, said method comprising:
(i) administering to said subject an antagonist which downregulates ACVR1B-mediated signalling for a time and under conditions sufficient for said antagonist to increase the sensitivity of said carcinoma to an agent which downregulates neoplastic cell growth, which agent is an alkylating agent or a platin agent; and
(ii) administering said agent to said subject for a time and under conditions sufficient to downregulate the neoplastic cell growth of said carcinoma.

In still yet another aspect there is provided a method for the treatment of a carcinoma in a subject, said method comprising:
(i) administering to said subject an antagonist of the functionality of each of GDF11 and activin A for a time and under conditions sufficient for said antagonist to increase the sensitivity of said carcinoma to an agent which downregulates neoplastic cell growth, which agent is an alkylating agent or a platin agent; and
(ii) administering said agent to said subject for a time and under conditions sufficient to downregulate the neoplastic cell growth of said carcinoma.

In a further aspect there is provided a method for the treatment of a neoplastic condition in a subject, said method comprising:
(i) administering an effective amount of follistatin to said subject for a time and under conditions sufficient for said follistatin to increase the sensitivity of a neoplastic cell to an agent which downregulates neoplastic cell growth, which agent is an alkylating agent or a platin agent; and
(ii) administering an effective amount of said agent to said subject for a time and under conditions sufficient to downregulate said neoplastic cell growth.

In yet another further aspect there is provided a method for the treatment of neoplastic condition in a subject, said method comprising:
(i) administering follistatin to said subject for a time and under conditions sufficient for said follistatin to increase the sensitivity of a neoplastic cell to a platin; and
(ii) administering said platin agent to said subject for a time and under conditions sufficient to downregulate said neoplastic cell growth;
wherein said platin is selected from:
   cisplatin
   carboplatin
   oxaliplatin
   satraplatin
   picoplatin
   Nedaplatin
   Triplatin
   Lipoplatin.

In one embodiment, said alkylating agent is a classical akylating agent or a non-classical alkylating agent.

In another embodiment, said alkylating agent is a Nitrogen mustard, nitrosoureas or alkyl sulfonate.

In still another embodiment, said alkylating agent is selected from:
Cyclophosphamide
Mechlorethamine or mustine (HN2) (trade name Mustargen)
Uramustine or uracil mustard
Melphalan
Chlorambucil
Ifosfamide
Bendamustine
Carrmustine
Lomustine
Streptozocin
Busulfan
procarbazine
altretamine
dicarbazine, mitozolamide and temozolomide.

In still another aspect there is provided a method for the treatment of neoplastic condition in a subject, said method comprising:
(i) administering follistatin to said subject for a time and under conditions sufficient for said follistatin to increase the sensitivity of a neoplastic cell to an alkylating agent; and
(ii) administering said alkylating agent to said subject for a time and under conditions sufficient to downregulate said neoplastic cell growth.

In a related aspect there is provided a method for the treatment of a neoplastic condition in a subject, said method comprising:
(i) administering to said subject an antagonist of the functionality of ACVR1B and TGFBR1 for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

Yet another aspect of the present invention is directed to the use of:
(i) an antagonist of ACVR1B-mediated signalling; and
(ii) an alkylating agent or a platin agent

in the manufacture of a medicament for the treatment of a neoplastic condition in a subject wherein:
(i) administering said antagonist to said subject downregulates ACVR1B-mediated signalling and increases the sensitivity of a neoplastic cell to an agent which downregulates neoplastic cell growth, which agent is a platin agent or alkylating agent; and
(ii) administering said agent to said subject for a time and under conditions sufficient to downregulates said neoplastic cell growth.

Yet another aspect of the present invention is directed to the use of:
(i) an antagonist of GDF11 and activin A; and
(ii) an alkylating agent or a platin agent

in the manufacture of a medicament for the treatment of a neoplastic condition in a subject wherein:
(i) administering said antagonist to said subject increases the sensitivity of a neoplastic cell to an agent which downregulates neoplastic cell growth, which agent is a platin agent or an alkylating agent; and
(ii) administering said agent to said subject for a time and under conditions sufficient to downregulates said neoplastic cell growth.

In a related aspect there is provided a method of reducing nephrotoxicity in a patient undergoing treatment with a chemotherapy agent, said method comprising administering to said patient an antagonist which downregulates ACVR1B-mediated signalling.

In another aspect, there is provided a method of reducing nephrotoxicity in a patient undergoing treatment with a chemotherapy agent, said method comprising administering to said patient an antagonist of the functionality of GDF11 and Activin A.

In still another aspect there is provided a method of reducing nephrotoxicity in a patient undergoing treatment with an alkylating agent or a platin agent, said method comprising administering to said patient an antagonist which downregulates ACVR1B-mediated signalling.

In yet another embodiment, there is provided a method of reducing nephrotoxicity in a patient undergoing treatment with an alkylating agent or a platin agent, said method comprising administering to said patient an antagonist of the functionality of GDF11 and Activin A.

In still yet another aspect there is provided the use of an antagonist which downregulates ACVR1B-mediated signalling in the manufacture of a medicament for a condition characterised by treatment with a chemotherapy agent.

In another aspect, there is provided the use of an antagonist of GDF11 and Activin A functionality in the manufacture of a medicament for a condition characterised by treatment with a chemotherapy agent.

### BRIEF DESCRIPTION OF THE DRAWINGS

FIG. 1 is a graphical representation of the percentage survival following treatment with increasing concentrations of carboplatin in chemoresistant A549 lung adenocarcinoma cell line compared to the chemosensitive SCLC cell line, LX22CL. The reported range of peak plasma concentrations for carboplatin in humans during treatment is indicated by the dotted lines. Data presented as the mean of 4 independent biological replicates±SEM.

FIG. 2 is confocal immunofluorescence images of the DNA damage response in A549 lung adenocarcinoma cells treated with increasing concentrations of carboplatin for 24 hours. Cells were stained for γH2AX, a marker of DNA damage, and the DNA counterstain DAPI. Scale bar=5 μm.

FIG. 3 is a schematic overview of the synthetic lethal whole genome siRNA screen. Synthetic lethal hits from the screen were further analysed via deconvolution, network analysis and orthogonal RNAi.

FIG. 4 is a graphical representation of the sensitization index of genes as determined from the synthetic lethal screen of the whole genome. Data shown in red dots makes up the top 5% of hits and can be analysed further for pathway analysis. Data is the ranking of genes against a sensitization index determined by fold change difference between vehicle and platinum treatments (n=4 for each treatment).

FIG. 5 is a schematic representation depicting the pathway analysis network of high confidence hits from the whole genome synthetic lethal screen, performed using the ClueGo plugin of the cytoscape network platform.

FIG. 6 is a graphical representation of the in vitro validation of TGFBR1 and ACVR1B as a potential therapeutic target for sensitization. Viability of A549 lung adenocarcinoma cells pre-treated with a non-targeting (NT) siRNA or specific siRNA targeting the receptors TGFBR1 or ACVR1B or the TGFβ superfamily ligands TGFB1, GDF11, INHBA or INHBB, followed by treatment with a range of carboplatin concentrations in vitro. Data presented as the mean of 4 independent biological replicates±SEM.

FIG. 7 is a graphical representation of the effects of TGFBR1 and ACVR1B pathway components knockdown in HEK293 immortalised renal epithelial cells. HEK293 cells were pre-treated with a non-targeting (NT) siRNA or specific siRNA targeting the receptors TGFBR1 or ACVR1B or the TGFβ superfamily ligands TGFB1, GDF11, INHBA or INHBB, followed by treatment with a range of carboplatin concentrations in vitro. Data presented as the mean of 4 independent biological replicates±SEM.

FIG. 8 is a graphical representation of the in vitro validation of targeting both TGFBR1 and ACVR1B receptors for sensitization using the small molecule inhibitor SB-505124. (A) Viability of chemoresistant adenocarcinoma cell lines A549 (top panel) and NCI-H358 (bottom panel) pre-treated with either SB-505124 or vehicle, followed by treatment with a range of carboplatin concentrations in vitro respectively. (B) Western Blots showing the relative levels of p-TAK 1 in A549 and NCI-H358 lung adenocarcinoma pre-treated with either SB-505124 or vehicle, followed by treatment with a range of carboplatin concentrations in vitro.

FIG. 9 is a graphical representation of the in vitro validation of targeting both TGFBR1 and ACVR1B receptors using SB-505124 for sensitisation of A549 lung adenocarcinoma cells in response to two other alkylating agents, busulfan and cisplatin. (A) Cells pre-treated with either SB-505124 or vehicle, followed by treatment with a range of busulfan concentrations in vitro. (B) Cells pre-treated with either SB-505124 or vehicle, followed by treatment with a range of cisplatin concentrations in vitro. Data presented as the mean of 4 independent biological replicates±SEM.

FIG. 10 is a graphical representation depicting (A) tumour volume and (B) survival of athymic nude mice bearing A549 flank xenografts. Mice were culled once the tumour size exceeded 500 mm³. (A) Growth of A549 flank xenografts in mice treated with combinations of vehicle control, FST (2 mg) on days −1, +1, +3 and +5, or carboplatin (Pt) 60 mg/kg on day 0. (B) Kaplan-Meier analysis of survival to ethical endpoint of mice from the same experiment #P<0.001 compared to mice treated with Vehicle+Pt, determined using the Log-rank (Mantel-Cox) test.

FIG. 11 is a graphical representation depicting platinum induced nephrotoxicity in mice. (A) A graphical representation of the treatment regimen where mice were treated with combinations of vehicle control, 5 mg/kg cisplatin on day 0, and/or 2 μg FST on day −1 and +1. (B) Graphical representation of plasma concentrations of urea and creatinine in treatment groups. (C) Macroscopic appearance of kidneys at day 9. Scale bar=2.5 mm N=5, mean±SEM. ***=P<0.001, **=P<0.01, One-Way ANOVA/Bonferonni.

FIG. 12 is a heat map depicting expression of mRNAs encoding ligands and receptors belonging to the TGFβ superfamily in the TCGA lung adenocarcinoma study capturing 230 tumours. Note that the differential gene expression is not a direct indicative of genes that confer platinum resistance.

FIG. 13 is a graphical representation of the association between TGFβ superfamily gene expression in lung adenocarcinoma and overall survival in the KMPlot lung adenocarcinoma dataset. Note that TGFB1 shows the only strong correlation between high levels of gene expression with higher probability of survival. TGFBR1

### DETAILED DESCRIPTION OF THE INVENTION

The present invention is predicated, in part, on the determination that antagonising ACVR1B-mediated signalling, and optionally TGFBR1-mediated signalling, in a neoplastic cell selectively increases the sensitivity of that cell, but not non-neoplastic cells, to platin or alkylating agent based chemotherapy. Still further, in patients where resistance to such therapy either pre-exists or has been induced to occur, the antagonism of ACVR1B-mediated signalling enables reversal of the resistant state such that chemotherapy can be effectively used. It has still further been determined that antagonising the functionality of each of GDF11 and activin A provides unexpectedly superior efficacy relative to antagonising other ligands or combinations of ligands which function as part of the ACVR1B or TGFBR1 signalling pathways.

The determinations of the present invention have enabled the development of a treatment regime which is highly effective, thereby facilitating minimisation both of the duration of the treatment protocol and the concentration of chemotherapy which is required to be administered. Accordingly, aside from the improved therapeutic outcomes, the side effects experienced by the patient are reduced. To this end, the further determination that antagonising ACVR1B-mediated signalling also reduces kidney and bone marrow toxicity which otherwise occurs and which is a serious side effect of chemotherapy, renders this method still more desirable. This development therefore now provides a realistic alternative to current neoplastic treatment regimes wherein both improved efficacy and reduced side effects are achievable.

Accordingly, one aspect of the present invention is directed to a method for the treatment of a neoplastic condition in a subject, said method comprising:
(i) administering to said subject an antagonist which downregulates ACVR1B-mediated signalling for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

In one embodiment, both ACVR1B and TGFBR1-mediated signalling are downregulated.

In a further aspect the present invention is directed to a method for the treatment of a neoplastic condition in a subject, said method comprising:
(i) administering to said subject an antagonist of the functionality of each of GDF11 and activin A for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

Reference to a “neoplastic condition” should be understood as a reference to a condition characterised by the presence of development of encapsulated or unencapsulated growths or aggregates of neoplastic cells. Reference to a “neoplastic cell” should be understood as a reference to a cell exhibiting abnormal growth. The term “growth” should be understood in its broadest sense and includes reference to enlargement of neoplastic cell size as well as proliferation.

The phrase “abnormal growth” of “neoplastic cell growth” in this context is intended as a reference to cell growth which, relative to normal cell growth, exhibits one or more of an increase in individual cell size and nuclear/cytoplasmic ratio, an increase in the rate of cell division, an increase in the number of cell divisions, a decrease in the length of the period of cell division, an increase in the frequency of periods of cell division or uncontrolled proliferation and evasion of apoptosis. Without limiting the present invention in any way, the common medical meaning of the term “neoplasia” refers to “new cell growth” that results as a loss of responsiveness to normal growth controls, eg. to neoplastic cell growth. Neoplasias include “tumours” which may be benign, pre-malignant or malignant. The term “neoplasm” should be understood as a reference to a lesion, tumour or other encapsulated or unencapsulated mass or other form of growth or cellular aggregate which comprises neoplastic cells.

The term “neoplasm”, in the context of the present invention should be understood to include reference to all types of cancerous growths or oncogenic processes, metastatic tissues or malignantly transformed cells, tissues or organs irrespective of histopathologic type or state of invasiveness.

The term “carcinoma” is recognised by those skilled in the art and refers to malignancies of epithelial or endocrine tissues including respiratory system carcinomas, gastrointestinal system carcinomas, genitourinary system carcinomas, testicular carcinomas, breast carcinomas, prostate carcinomas, endocrine system carcinomas and melanomas. Exemplary carcinomas include those forming from tissue of the breast. The term also includes carcinosarcomas, e.g. which include malignant tumours composed of carcinomatous and sarcomatous tissues. An “adenocarcinoma” refers to a carcinoma derived from glandular tissue or in which the tumour cells form recognisable glandular structures.

The neoplastic cells comprising the neoplasm may be any cell type, derived from any tissue, such as an epithelial or non-epithelial cell. Reference to the terms “malignant neoplasm” and “cancer” and “carcinoma” herein should be understood as interchangeable.

The term “neoplasm” should be understood as a reference to a lesion, tumour or other encapsulated or unencapsulated mass or other form of growth or cellular aggregate which comprises neoplastic cells. The neoplastic cells comprising the neoplasm may be any cell type, derived from any tissue, such as an epithelial or non-epithelial cell. Examples of neoplasms and neoplastic cells encompassed by the present invention include, but are not limited to central nervous system tumours, retinoblastoma, neuroblastoma, paediatric tumours, head and neck cancers (e.g. squamous cell cancers), breast and prostate cancers, lung cancer (both small and non-small cell lung cancer), kidney cancers (e.g. renal cell adenocarcinoma), oesophagogastric cancers, hepatocellular carcinoma, pancreaticobiliary neoplasias (e.g. adenocarcinomas and islet cell tumours), colorectal cancer, cervical and anal cancers, uterine and other reproductive tract cancers, urinary tract cancers (e.g. of ureter and bladder), germ cell tumours (e.g. testicular germ cell tumours or ovarian germ cell tumours), ovarian cancer (e.g. ovarian epithelial cancers), carcinomas of unknown primary, human immunodeficiency associated malignancies (e.g. Kaposi's sarcoma), lymphomas, malignant melanomas, sarcomas, endocrine tumours (e.g. of thyroid gland), mesothelioma and other pleural or peritoneal tumours, neuroendocrine tumours and carcinoid tumours.

In another embodiment, said neoplastic condition is a malignant neoplastic condition, in particular a carcinoma.

Accordingly, in one embodiment there is provided a method for the treatment of a carcinoma in a subject, said method comprising:
(i) administering to said subject an antagonist which downregulates ACVR1B-mediated signalling for a time and under conditions sufficient for said antagonist to increase the sensitivity of said carcinoma to an agent which downregulates neoplastic cell growth, which agent is an alkylating agent or a platin agent; and
(ii) administering said agent to said subject for a time and under conditions sufficient to downregulate the neoplastic cell growth of said carcinoma.

In another embodiment, both ACVR1B and TGFBR1-mediated signalling are downregulated.

In still another embodiment there is provided a method for the treatment of a carcinoma in a subject, said method comprising:
(i) administering to said subject an antagonist of the functionality of each of GDF11 and activin A for a time and under conditions sufficient for said antagonist to increase the sensitivity of said carcinoma to an agent which downregulates neoplastic cell growth, which agent is an alkylating agent or a platin agent; and
(ii) administering said agent to said subject for a time and under conditions sufficient to downregulate the neoplastic cell growth of said carcinoma.

In another embodiment, said carcinoma is an adenocarcinoma. In yet another embodiment, said carcinoma or adenocarcinoma is of the lung. In still another embodiment, said lung adenocarcinoma is small cell lung cancer.

In still yet another embodiment, said neoplastic condition is characterised by neoplastic cells which exhibit defective DNA repair mechanisms.

According to this embodiment there is provided a method for the treatment of a neoplastic condition in a subject, which neoplastic condition is characterised by neoplastic cells that exhibit defective DNA repair mechanisms, said method comprising:
(i) administering to said subject an antagonist which downregulates ACVR1B-mediated signalling for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate the neoplastic cell growth.

In another embodiment, both ACVR1B and TGFBR1-mediated signalling are downregulated.

In yet another embodiment there is provided a method for the treatment of a neoplastic condition in a subject, which neoplastic condition is characterised by neoplastic cells that exhibit defective DNA repair mechanisms, said method comprising: 0

(i) administering to said subject an antagonist of the functionality of each of GDF11 and activin A for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate the neoplastic cell growth.

In another embodiment, said neoplastic condition is malignant, in particular metastatic. In yet another embodiment said neoplastic condition is a carcinoma or an adenocarcinoma.

In still another embodiment, said neoplastic condition is non-small cell lung cancer, breast cancer, pancreatic cancer, mesothelioma, lymphoma, testicular cancer, ovarian cancer, small cell carcinoma, colorectal cancer, oral cancer, head and neck cancer, cervical cancer, bladder cancer or epithelial cancer.

In yet still another embodiment, said neoplastic condition is non-small cell lung cancer, testicular cancer, ovarian cancer, gastric cancer or bladder cancer.

As detailed hereinbefore, it has been determined that the sensitivity of neoplastic cells to chemotherapy, in particular neoplastic cells characterised by defective DNA repair mechanisms, can be rendered more sensitive to an alkylating agent or platin agent where that cell has been specifically exposed to an antagonist of ACVR1B-, and optionally TGFBR1-mediated signalling. Without limiting the present invention to any one theory or mode of action, ACVR1B and TGFBR1 are both type I receptors to which selected TGFβ superfamily ligands bind. The activin type I receptors transduce signals for, inter alia, Anti-müllerian hormone (AMH), bone morphogenetic proteins (BMPs), and nodal. Like ACVR1B, TGFBR1 is also bound by a range of ligands, including Mothers against decapentaplegic homolog 7, FKBP1A, TGF beta 1, caveolin 1, FNTA, PPP2R2A, TGFβ receptor 2, Endoglin, Heat shock protein 90 kDa alpha (cytosolic), member A1 and STRAP.

Reference to “ACVR1B” and “TGFBR1” should be understood as a reference to all forms of ACVR1B and TGFBR1 and to functional fragments and variants thereof. It should also be understood to include reference to any isoforms which may arise from the alternative splicing of the encoding genes and polymorphic forms of these molecules.

Reference to “ACVR1B- or TGFBR1-mediated signalling” should be understood as reference to an intracellular signalling pathway which utilises one of these receptors. As detailed hereinbefore, each of these receptors is bound by a range of ligands from the TGFβ superfamily Ligands that signal through ACVR1B include, but are not limited to, activin A, activin B, GDF 1, GDF3, GDF8, GDF9, GDF10, GDF11 and Nodal. Ligands that signal through TGFBR1 include, but are not limited to, TGFβ1, TGFβ2, TGFβ3 and GDF1, GDF3, GDF8, GDF9, GDF11. In terms of the present invention, it has been determined that antagonising, in neoplastic cells, the functioning of the signalling pathways which utilise these receptors will act to sensitise (or re-sensitise) the subject cell to the toxicity of an alkylating agent or a platin agent. By “antagonising” and “antagonist” is meant that the subject signalling is downregulated, that is, prevented, reduced or otherwise inhibited. Said downregulation may be a partial reduction in signalling activity or a complete cessation of the signalling. In one embodiment, the subject downregulation is effected transiently for a period of time sufficient to enable the platin or radiation treatment step to be administered.

As detailed hereinbefore, it has been unexpectedly determined that the sensitivity of neoplastic cells to chemotherapy and radiation treatment, in particular neoplastic cells characterised by defective DNA repair mechanisms, can be rendered particularly sensitive to an alkylating agent or platin agent where that cell has been exposed to an antagonist of GDF11 and activin A. Bearing in mind that prior art methods have consistently focussed on modulating the activity of a single agent thought to be involved in cancer cell resistance and, prior to the advent of the present invention, have been unsuccessful in terms of increasing chemosensitivity and/or reducing acquired chemoresistance at a level which would be clinically useful, the findings of this aspect the present invention are highly significant and unexpected.

Without limiting the present invention to any one theory or mode of action, this aspect of the present invention is directed to targeting an antagonist to the molecules GDF11 and activin A, which both exhibit functionality as extracellular signalling molecules, in order to reduce their level of functionality. Reference to “activin A” should be understood as a reference to all forms of these molecules. Activin A is a homodimer of the activin β_{A} subunit. “Activin β_{A}” is also interchangeably referred to as “activin β_{A} subunit”. Reference to “activin A” should also be understood to include reference to any isoforms which may arise from alternative splicing of activin β_{A} mRNA or mutant or polymorphic forms of activin β_{A}. Reference to “activin A” is not intended to be limiting and should be read as including reference to all forms of activin A (and its subunit monomers) including any precursor forms which may be generated, and any activin A protein, whether existing as a monomer, multimer or fusion protein. Reference to “GDF11” should similarly be understood as a reference to all forms of this molecule. In this regard, as for activin A, this molecule is well known to the skilled person. GDF11 (also known as BMP11) is described in Gamer, L. W., N. M. Wolfman, et al. (1999). Developmental biology 208: 222-232 and the sequence of the precursor protein is shown in SEQ ID NO:1 and the mature protein in SEQ ID NO:2.

By “antagonising” and “antagonist” is meant that the functionality of the subject ligands (GDF11 and Activin A) are down-regulated, that is, prevented, reduced or otherwise inhibited. Said down-regulation may be a partial reduction in functionality (eg. a reduction in its ability to interact with its receptor and thereby facilitate intra-cellular signalling) or a complete cessation of functioning. In one embodiment, the subject downregulation is effected transiently for a period of time sufficient to enable the chemotherapy treatment step to be administered. It should be understood that one may utilise a single molecule which can antagonise both ligands or two separate antagonists which are separately directed to each ligand. Accordingly, reference to an “antagonist” of GDF11 or Activin A should be understood as reference to the use of either a common antagonist or two different antagonists.

The ACVR1B and TGFBR1 signalling pathways are well understood. Accordingly, selecting a means by which said downregulating in signalling can be achieved would be a matter of routine procedure for the skilled person. Any suitable means may be utilised including, but not limited to:
(i) downregulating the absolute levels of the components of the ACVR1B- or TGFBR1-mediated signalling pathway such that a reduced level of these molecules are available for activation and/or to interact with downstream targets. In this regard, the extracellular ligands of ACVR1B and TGFBR1 should be understood to be included in the definition of the “components” which form part of the subject signalling pathways;
(ii) antagonising the components of the ACVR1B- or TGFBR1-mediated signalling pathway such that the functional effectiveness of any one or more of these molecules is decreased. To this end, reference to an “antagonist” should be understood as a reference to an agent which interacts with a component of the ACVR1B or TGFBR1 signalling pathway and prevents, reduces or otherwise inhibits its functionality.

In terms of achieving the downregulation of ACVR1B- or TGFBR1-mediated signalling, means for achieving this objective would be well known to the person of skill in the art and include, but are not limited to:
(i) introducing into a cell a proteinaceous or non-proteinaceous molecule which antagonises transcription or translation of a gene, wherein this gene may be an ACVR1B or TGFBR1 signalling pathway component or some other gene or gene region (e.g. promoter region) which directly or indirectly modulates the expression of an ACVR1B or TGFBR1 signalling pathway component. For example, siRNA directed to the ACVR1B or TGFBR1 genes, or some other gene which directly or indirectly modulates the expression of the components of ACVR1B- or TGFBR1-mediated signalling pathways may be used; 0

(ii) introducing a proteinaceous or non-proteinaceous molecule which functions as an antagonist of the functioning of any of the components of the ACVR1B or TGFBR1 signalling pathway, such as an antagonist of one or more of the ligands of ACVR1B or TGFBR1.

In terms of achieving the antagonism of each of GDF11 and activin A, means for achieving this objective would be well known to the person of skill in the art and include, but are not limited to:
(i) introducing into a cell a proteinaceous or non-proteinaceous molecule which antagonises transcription or translation of a gene which encodes GDF11 or activin A or some other gene or gene region (e.g. promoter region) which directly or indirectly modulates the expression of GDF11 or activin A. For example, siRNA directed to the GDF11 or activin A genes, or some other gene which directly or indirectly modulates the expression of GDF11 or activin A may be used;
(ii) introducing a proteinaceous or non-proteinaceous molecule which functions as an antagonist of the functioning of GDF11 or activin A.

The proteinaceous molecules described above may be derived from any suitable source such as natural, recombinant or synthetic sources and include fusion proteins, variants or molecules which have been identified following, for example, natural product screening. In another example, one may utilize a genetically modified variant, such as a modified activin or other ligand molecule in which the prodomain has been modified to create an activin antagonist. The reference to non-proteinaceous molecules may be, for example, a reference to a nucleic acid molecule or it may be a molecule derived from natural sources, such as for example natural product screening, or may be a chemically synthesised molecule. The present invention contemplates small molecules capable of acting as antagonists. Antagonists may be any compound capable of blocking, inhibiting or otherwise preventing the subject signalling pathway components from carrying out its normal biological function. Antagonists include monoclonal antibodies and antisense nucleic acids which prevent transcription or translation of genes or mRNA in mammalian cells, such as the ACVR1B, TGFBR1, GDF11 and Activin A genes, as exemplified herein. Modulation of expression may also be achieved utilising antigens, RNA, ribosomes, DNAzymes, aptamers, antibodies or molecules suitable for use in cosuppression. Suitable antisense oligonucleotide sequences (single stranded DNA fragments) may be created or identified by their ability to suppress the expression of the target component. The production of antisense oligonucleotides for a given protein is described in, for example, Stein and Cohen, 1988 (Cancer Res 48:2659-2668) and van der Krol et al., 1988 (Biotechniques 6:958-976). Antagonists also include any other molecule that prevents the subject components from functioning, such as molecules which prevent the ACVR1B or TGFBR1 ligands interacting with their receptor.

In the context of antibodies, the present invention envisages the use of any suitable form of antibody including catalytic antibodies or derivatives, homologues, analogues or mimetics of said antibodies. Such antibodies may be monoclonal or polyclonal and may be selected from naturally occurring antibodies be specifically raised to a component in issue. Alternatively, fragments of antibodies may be used such as Fab fragments or Fab′₂ fragments. Furthermore, the present invention extends to recombinant and synthetic antibodies and to antibody hybrids. A “synthetic antibody” is considered herein to include fragments and hybrids of antibodies. The subject components can also be used to screen for naturally occurring antibodies.

Both polyclonal and monoclonal antibodies are obtainable by immunization with the component or derivative, homologue, analogue, mutant, or mimetic thereof and either type is utilizable therapeutically. The methods of obtaining both types of sera are well known in the art. Polyclonal sera are less preferred but are relatively easily prepared by injection of a suitable laboratory animal with an effective amount of the component, or antigenic parts thereof, collecting serum from the animal, and isolating specific sera by any of the known immunoadsorbent techniques. Although antibodies produced by this method are utilizable, they are generally less favoured because of the potential heterogeneity of the product.

The use of monoclonal antibodies is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman 1981, Basic Facts about Hybridomas, in Compendium of Immunology Vol II, ed. by Schwartz; Kohler and Milstein 1975, Nature 256:495-499; Kohler and Milstein 1976, Eur J Immun 6:511-519).

Preferably, an antibody used in the present invention specifically binds the component in issue. By “specifically binds” is meant high avidity and/or high affinity binding of an antibody to a specific antigen. Antibody binding to its epitope on this specific component is stronger than binding of the same antibody to any other epitope, particularly those that may be present in molecules in association with, or in the same sample, as the specific component of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g. by use of appropriate controls.

The proteinaceous and non-proteinaceous molecules referred to, above, are herein collectively referred to as “antagonists”. To the extent that it is sought to decrease ACVR1B or TGFBR1 activity, said antagonist is preferably:
(i) Follistatin. This may be administered either as a protein or its overexpression may be induced in vivo such as via the adenovirus mediated system described by Takabe et al. 2003 (Hepatology 38:1107-1115).
(ii) Any agent that upregulates the expression or functioning of the α subunit of inhibin. The α subunit can dimerise with the β subunits of activin to form inhibin, thereby effectively downregulating activin levels.
(iii) Inhibin. This molecule can bind to β-glycan and inhibit the actions of activin via its receptor. See for example the mechanism described by Xu et al. 1995 (J Biol Chem 270:6308-6313) or the use of the Smad7 antagonist (Bernard et al. 2004, Molecule Endocrinol 18:606-623).
(iv) Neutralising antibodies directed to ligands that signal through ACVR1B and TGFBR1. For example, as described in Poulaki et al. 2004 (Am J Pathol 164:1293-1302) and Whittemore and Song 2003 (Biochem & Biophys Res. Comm 300(4):965-971.
(v) Mutated ligands which inhibit native ligands from binding to ACVR1B and TGFBR1. For example, as described in Harrison et al. 2004, (J Biol Chem 279:28036-28044), or modifications of the prodomain of the ligands that signal through ACVR1B or TGFBR1(see Makanji Y et al. 2011 Generation of a specific activin B antagonist by modification of the activin B propeptide. Endocrinol 152:3758-3768).
(vi) Transfection or treatment with a mutant activin or TGFβ receptor which prevents normal signalling through ACVR1B or TGFBR1 or a soluble activin receptor or TGFβ which acts as a competitive inhibitor. See for example, the system described by Maeshima et al. 2004 (Endocrinology 145:3739-3745), Morrison, B. M., J. L. Lachey, et al. 2009 (Experimental neurology 217: 258-268), Dussiot, M., T. T. Maciel, et al. 2014 (Nature medicine 20(4): 398-407) and Suragani, R. N. V. S., S. M. Cadena, et al. 2014 (Nature medicine 20(4): 408-414).
(vii) The Cripto protein. This protein is required for nodal signalling. However, it specifically binds to activin and inhibits it's signalling (Gray et al. 2003).
(viii) An antibody directed to ACVR1B (also known as ALK4) or TGFBR1, which blocks binding of a naturally occurring ligand, or an antibody directed to the ligand itself.
(ix) siRNA designed to downregulate the transcription of the ACVR1B or TGFBR1 genes.
(x) Beta-glycan and BAMBI (membrane-bound antagonists) and Follistatin-like 3. These molecules are antagonists of activin signalling.
(xi) Small molecule inhibitors of ACVR1B or TGFBR1 including, but not limited to: SB-431542, SB-505124, A83-01, D-4476, GW-788388, LY-36497, RepSox, SB-525334, SD-208, A-77-01, SM16.
(xii) Any mutants of ACVR1B or TGFBR1 that act to bind to the ligands but do not effect signalling.
(xiii) Soluble receptors or ligand traps including but not limited to, Sotatercept, RAP-011, ACE-011, RAP-031, ACE-041, ACE-031.
(xiv) Soluble ACVR1B or TGFBR1 molecules.

In one embodiment, said antagonist is follistatin or an antagonist of the ACVR1B receptor, such as an antibody, soluble receptor or siRNA.

The proteinaceous and non-proteinaceous molecules referred to, above, are herein collectively referred to as “antagonists”. To the extent that it is sought to decrease GDF11 and activin A activity, said antagonists may be selected from:
(i) Follistatin. This may be administered either as a protein or its overexpression may be induced in vivo such as via the adenovirus mediated system described by Takabe et al. 2003 (Hepatology 38:1107-1115).
(ii) Any agent that upregulates the expression or functioning of the α subunit of inhibin. The α subunit can dimerise with the β subunits of activin to form inhibin, thereby effectively downregulating activin levels.
(iii) Inhibin. This molecule can bind to β-glycan and inhibit the actions of activin via its receptor. See for example the mechanism described by Xu et al. 1995 (J Biol Chem 270:6308-6313) or the use of the Smad7 antagonist (Bernard et al. 2004, Molecule Endocrinol 18:606-623).
(iv) Neutralising antibodies directed to GDF11 or activin A. For example, as described in Poulaki et al. 2004 (Am J P athol 164:1293-1302) and Whittemore and Song 2003 (Biochem & Biophys Res. Comm 300(4): 965-971).
(v) GDF11 or activin A mutants which inhibit the native molecule from binding to its receptor. For example, as described in Harrison et al. 2004, (J Biol Chem 279:28036-28044), or modifications of the prodomain of GDF11 or activin A propeptide
(vi) Transfection or treatment with a mutant of GDF11 or activin A receptor which prevents normal signalling, or a soluble receptor which acts as a competitive inhibitor. See for example, the systems described by Maeshima et al. 2004 (Endocrinology 145:3739-3745), Morrison, B. M., J. L. Lachey, et al. 2009 (Experimental neurology 217: 258-268), Dussiot, M., T. T. Maciel, et al. 2014 (Nature medicine 20(4): 398-407) and Suragani, R. N. V. S., S. M. Cadena, et al. 2014 (Nature medicine 20(4): 408-414).
(vii) A GDF11 or activin A antisense oligonucleotide (siRNA) designed to downregulate the transcription of GDF11 or activin A, for example, Yang, Z., J. Zhang, et al. 2008 The Journal of Gene Medicine 10(8): 825-833).
(viii) The Cripto protein. This protein is required for nodal signalling. However, it specifically binds to activin and inhibits it's signalling (Gray et al. 2003).
(ix) Soluble receptors or ligand traps which can be used to competitively inhibit binding of the ligand to the cellular receptor. Examples of such receptors include, but are not limited to Sotatercept (ACE-011), RAP-011, RAP-031, ACE-041, ACE-031, ACE-083, ACE-536. An Example of a ligand trap can be found in WO2011/020045.

In a preferred embodiment, the subject antagonist is follistatin, which has been shown to antagonise both GDF11 and activin A. However, it would be appreciated by the person of skill in the art that one may also elect to use two different antagonists which, together, antagonise each of GDF11 and activin A. For example, one may elect to use two antibodies, each specifically directed to one of GDF11 and activin A. Alternatively, there may be antagonists that interact with both of these ligands and therefore the overall number of antagonists which are required to be used is only one.

In this regard, reference to “follistatin” should be read as including reference to all forms of follistatin including, by way of example, the three protein cores and six molecular weight forms which have been identified as arising from the alternatively spliced mRNAs FS315 and FS288. Accordingly, it should also be understood to include reference to any isoforms which may arise from alternative splicing of follistatin mRNA or mutant or polymorphic forms of follistatin. It should still further be understood to extend to any protein encoded by the follistatin gene, any subunit polypeptide, such as precursor forms which may be generated, and any follistatin protein, whether existing as a monomer, multimer or fusion protein. An analogous definition applies to “inhibin”.

Other forms of follistatin which are suitable for use in the present invention include:
(i) Wild-type follistatin (FS), comprising an N-terminal domain (ND) followed by three follistatin domains (FSD1, FSD2 and FSD3) with a heparin-binding sequence located in FSD1 (amino acid sequence positions 72-86), and all known isoforms thereof.
(ii) Wild-type follistatin-like 3 protein (FSTL3), which is also known as follistatin-related gene product (FLRG) and follistatin-related protein (FSRP), comprising an N-terminal domain (N3D) followed by two follistatin-like 3 domains (FS3D1 and FS3D2), and all known isoforms thereof.
(iii) Follistatin analogue having the structure ND-FSD1-FSD2 (i.e. wild-type minus FSD3).
(iv) Analogues of (i) and (iii) above with FSD1 substituted by FSD1′, where FSD1′ represents FSD1 with heparin-binding site removed.
(v) Analogues of (i) and (iii) above with FSD1 substituted by FSD1*, where FSD1* represents FSD1 with sequence prior to and including the heparin-binding sequence removed.
(vi) Hybrid forms of (i) and (iii) above where at least one of the domains is substituted by a corresponding FSTL3 domain N3D, FS3D1 and FS3D2.
(vii) Hybrid forms of (ii) above where at least one of the domains is substituted by a corresponding FS domain ND, FSD1, FSD1′, FSD1* and FSD2.
(viii) Any of the above proteins modified by one or more deletions, insertions and/or mutations in ND, N3D, FSD1, FSD1′, FSD1*, FS3D1, FSD2, FS3D2, and FSD3 provided the modified protein functions as an antagonist to ACVR1B or TGFBR1 ligands.
(ix) Genetically modified forms of follistatin which have been modified to preferentially antagonize one or more ligands over other follistatin targets.

(Jennifer N. Cash, Elizabeth B. Angerman, Henry T. Keutmann, and Thomas B. Thompson 2012 Characterization of Follistatin-Type Domains and Their Contribution to Myostatin and Activin A Antagonism. Mol Endocrinol, 26(7):1167-1178; Henry T. Keutmann, Alan L. Schneyer and Yisrael Sidis 2004 The Role of Follistatin Domains in Follistatin Biological Action. Mol Endocrinol, 18(1):228-240).

Screening for the antagonists hereinbefore defined can be achieved by any one of several suitable methods including, but in no way limited to, contacting a cell comprising a gene which encodes a component of the ACVR1B, TGFBR1, GDF11 or Activin A signalling pathway or functional equivalent or derivative thereof with an agent and screening for the modulation of protein production or functional activity, modulation of the expression of a nucleic acid molecule or modulation of the activity or expression of a downstream cellular target. Detecting such modulation can be achieved utilising techniques such as Western blotting, electrophoretic mobility shift assays and/or the readout of reporters such as luciferases, CAT and the like.

It should be understood that the subject gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model particularly useful for screening for agents which down regulate activity, at either the nucleic acid or expression product levels. To the extent that a nucleic acid molecule is transfected into a cell, that molecule may comprise the entire gene of interest or it may merely comprise a portion of the gene such as the portion which regulates expression. For example, the promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream regulatory target. Yet another example includes component binding sites ligated to a minimal reporter.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the subject nucleic acid molecule or expression product itself.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound or otherwise associated with any other proteinaceous or non-proteinaceous molecules.

The term “expression” refers to the transcription and translation of a nucleic acid molecule. Reference to “expression product” is a reference to the product produced from the transcription and translation of a nucleic acid molecule.

A “variant” or “mutant” should be understood to mean molecules which exhibit at least some of the functional activity of the form of molecule (e.g. follistatin) of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of follistatin, for example, which exhibits similar and suitable functional characteristics to that of the follistatin which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods (e.g., Bunin et al. 1994, Proc Natl Acad Sci USA 91:4708-4712; DeWitt et al. 1993, Proc Natl Acad Sci USA 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in US. Pat. No. 5,763,263.

There is always widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

The present invention is also directed to useful aptamers. In one embodiment, an aptamer is a compound that is selected in vitro to bind preferentially to another compound (in this case the identified signalling pathway components). In one aspect, aptamers are nucleic acids or peptides. Random sequences can be readily generated from nucleotides or amino acids (naturally occurring and/or synthetically made) in large numbers but of course they need not be limited to these. In another aspect, the nucleic acid aptamers are short strands of DNA that bind protein targets, such as oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function (for example, see the following publications describing in vitro selection of aptamers: Klug et al. 1994, Mol Biol Rep 20:97-107; Wallis et al. 1995, Chem Biol 2:543-552; Ellington 1994, Curr Biol 4:427-429; Lato et al. 1995, Chem Biol 2:291-303; Conrad et al. 1995, Mol Divers 1:69-78; and Uphoff et al. 1996, Curr Opin Struct Biol 6:281-287).

Certain RNA inhibiting agents may be utilized to inhibit the expression or translation of messenger RNA (“mRNA”) that is associated with a phenotype of interest. Examples of such agents suitable for use herein include, but are not limited to, short interfering RNA (“siRNA”), ribozymes, aptamers, and antisense oligonucleotides.

In some instances, a range of 18-25 nucleotides is the most preferred size for siRNAs. siRNAs can also include short hairpin RNAs in which both strands of an siRNA duplex are included within a single RNA molecule. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the RNA or internally (at one or more nucleotides of the RNA).

In one embodiment, the RNA molecules contain a 3′ hydroxyl group. Nucleotides in the RNA molecules of the present invention can also comprise non-standard nucleotides, including non-naturally occurring nucleotides or deoxyribonucleotides. Collectively, all such altered RNAs are referred to as analogues of RNA. siRNAs of the present invention need only be sufficiently similar to natural RNA that it has the ability to mediate RNA interference (RNAi).

Methods for designing double stranded RNA to inhibit gene expression in a target cell are known (see, e.g., U.S. Pat. No. 6,506,559; Elbashir et al. 2002, Methods 26:199-213; Chalk et al. 2004, Biochem Biophys Res Commun 319:264-274; Cui et al. 2004, Comput Methods Programs Biomed 75:67-73; Wang et al. 2004, Bioinformatics 20:1818-1820). For example, design of siRNAs (including hairpins) typically follow known thermodynamic rules (see, e.g., Schwarz, et al. 2003, Cell 115:199-208; Reynolds et al. 2004, Nat Biotechnol. 22:326-330; Khvorova et al. 2003, Cell 115:209-216). Many computer programs are available for selecting regions of a sequence that are suitable target sites. These include programs available through commercial sources such as Ambion, Dharmacon, Promega, Invitrogen, Ziagen, and GenScript as well as non-commercial sources such as EMBOSS, The Wistar Institute, Whitehead Institute, and others.

For example, design can be based on the following considerations. Typically, shorter sequences, less than about 30 nucleotides are selected. The coding region of the mRNA is usually targeted. The search for an appropriate target sequence optionally begins 50-100 nucleotides downstream of the start codon, as untranslated region binding proteins and/or translation initiation complexes may interfere with the binding of the siRNA endonuclease complex. Some algorithms, e.g., based on the work of Elbashir et al. 2000 (Methods 26:199-213) search for a selected sequence motif and select hits, with approximately 50% G/C-content (30% to 70% has also worked). If no suitable sequences are found, the search is extended.

Other nucleic acids, e.g., ribozymes, antisense, can also be designed based on known principles. For example, Sfold (see, e.g., Ding, et al., Nucl Acids Res 32 Web Server issue, W135-W141;, Ding & Lawrence 2003, Nucl Acids Res 31:7280-7301; and Ding & Lawrence 2001, Nucl Acids Res 20:1034-1046) provides programs relating to designing ribozymes and antisense, as well as siRNAs.

In terms of performing the method of the present invention, an “effective amount” means an amount necessary to at least partly attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

It should also be understood that in terms of applying the method of the present invention, the method may be applied either to patients who are newly diagnosed with a neoplastic condition and are to undergo chemotherapy for the first time or it may be applied to patients who have previously undergone chemotherapy. These latter patients may either have not been successfully treated through to remission or they may have relapsed from a remissive state. In either situation, there may be benefit in treating those patients in accordance with the method of the present invention to increase their sensitivity to platinum or alkylating agent therapy. In yet another example, patients may previously have been treated with the method of the present invention but are nevertheless recommended to be treated again with this method. This may be useful, for example, where a patient presents with a new primary tumour which differs from the origin of the previously treated tumour.

In one embodiment, the subject antagonist is follistatin.

In accordance with this embodiment there is provided a method for the treatment of a neoplastic condition in a subject, said method comprising:
(i) administering an effective amount of follistatin to said subject for a time and under conditions sufficient for said follistatin to increase the sensitivity of a neoplastic cell to an agent which downregulates neoplastic cell growth, which agent is an alkylating agent or a platin agent; and
(ii) administering an effective amount of said agent to said subject for a time and under conditions sufficient to downregulate said neoplastic cell growth.

In one embodiment, said neoplastic condition is a malignant condition. In another embodiment said malignant condition is a carcinoma or an adenocarcinoma. In yet another embodiment, said neoplastic condition is metastatic.

In still another embodiment, said neoplastic condition is characterised by neoplastic cells which exhibit defective DNA repair mechanisms.

In still yet another embodiment, said neoplastic condition is lung cancer, testicular cancer, ovarian cancer or bladder cancer.

As detailed hereinbefore, the subject antagonist has been found to act to increase the sensitivity of neoplastic cells to a platin agent or alkylating agent. By “increase in sensitivity” is meant that subsequently to treatment with the agent, the neoplastic cell will undergo cytolysis (or other mechanism of cell death) more efficiently or effectively than an untreated neoplastic cell. Examples of increased sensitivity include, but are not limited to, the induction of cell death of a higher proportion of neoplastic cells than is achievable in the absence of sensitisation, the induction of neoplastic cell death using lower doses of platin agent or alkylating agent than normally used, the induction of cell death by a treatment protocol of a shortened duration relative to normal protocols or the induction of cell death of neoplastic cells which are either innately, or have developed, resistance to a platin or alkylating agent chemotherapy. It should be understood that not necessarily every single cell will exhibit increased sensitivity. However, a sufficient proportion of treated cells will have undergone the transition to increased sensitivity thereby leading to a therapeutic outcome which is improved relative to that of a patient who has not been treated in accordance with the method of the present invention.

The method of the present invention has been determined to selectively induce the increased sensitivity of neoplastic cells to alkylating or platin-based therapy. This is an extremely valuable finding since it means that non-neoplastic cells are more significantly protected from the chemotherapy than in the absence of this pre-treatment. Similarly, the determination that the method of the present invention reduces renal, neuro and bone marrow toxicity is also entirely unexpected and highly desirable.

In this regard, reference to “alkylating agents” should be understood as a reference to an alkylating antineoplastic agent. Without limiting the present invention to any one theory or mode of action, alkylation agents used in cancer treatment attach an alkyl group (CₙH₂ₙ₊₁) to DNA. The alkyl group is attached to the guanine base of DNA, at the number 7 nitrogen atom of the purine ring. These agents function by inducing DNA damage. More specifically, they stop neoplastic cell growth by crosslinking guanine nucleobases in DNA double-helix strands, directly attacking DNA. This makes the strands unable to uncoil and separate. As this is necessary in DNA replication, the cells can no longer divide. Alkylating agents are used to treat a range of cancers, however, like most chemotherapy drugs, are also toxic to normal cells, in particular cells that divide frequently, such as those in the gastrointestinal tract, bone marrow, testicles and ovaries. Most alkylating agents are also themselves carcinogenic. Accordingly, the prospect of improving neoplastic cell sensitivity to these drugs and thereby potentially enabling the administration of lower doses or shorter administration protocols is a highly attractive prospect.

Alkylating agents can be broadly classified as either dialkylating agents or monoalkylation agents. Dialkylating agents can react with two different 7-N-guanine residues, and, if these are in different strands of DNA, the result is cross-linkage of the DNA strands, which prevents uncoiling of the DNA double helix. If the two guanine residues are in the same strand, the result is termed limpet attachment of the drug molecule to the DNA. Busulfan is an example of a dialkylating agent: it is the methanesulfonate diester of 1,4-butanediol. Monoalkylating agents can react only with one 7-N of guanine. Alkylating agents can then be further classified as follows:
(i) Classical Alkylating Agents

Classical alkylating agents include those agents exhibiting true alkyl groups. They act by adding an alkyl group to DNA molecule and preventing its replication. The following three groups are almost always considered “classical”:
Nitrogen mustards
   Cyclophosphamide
   Mechlorethamine or mustine (HN2) (trade name Mustargen)
   Uramustine or uracil mustard
   Melphalan
   Chlorambucil
   Ifosfamide
   Bendamustine
Nitrosoureas
   Carmustine
   Lomustine
   Streptozocin
Alkyl sulfonates
   Busulfan

Thiotepa and its analogues are usually considered classical, but can be considered nonclassical.
(ii) Nonclassical alkylating agents

Certain alkylating agents are sometimes described as “nonclassical” and they include, but are not limited to:
procarbazine
altretamine
dicarbazine, mitozolomide and temozolomide.

Accordingly, in one embodiment, said alkylating agent is a classical alkylating agent or a non-classical alkylating agent.

In another embodiment, said alkylating agent is a Nitorgen mustard, nitrosoureas or alkyl sulfonate.

In still another embodiment, said alkylating agent is selected from:
Cyclophosphamide
Mechlorethamine or mustine (HN2) (trade name Mustargen)
Uramustine or uracil mustard
Melphalan
Chlorambucil
Ifosfamide
Bendamustine
Carrmustine
Lomustine
Streptozocin
Busulfan
procarbazine
altretamine
dicarbazine, mitozolamide and temozolomide.

Reference to “platin agent” should be understood as a reference to platinum-based antineoplastic drugs. Without limiting the present invention to any one theory or mode of action, platins are co-ordination complexes of platinum. They function by causing crosslinking of DNA as monoadduct, interstrand crosslinks, intrastrand crosslinks or DNA protein crosslinks Mostly they act on the adjacent N-7 position of guanine, forming 1, 2 intrastrand crosslink The resultant crosslinking inhibits DNA repair and/or DNA synthesis in cancer cells. Platinum-based antineoplastic agents are also sometimes described as “alkylating-like” due to similar effects of alkylating antineoplastic agents, although they do not exhibit an alkyl group. The mean dose-limiting side effect of cancer treatment with platinum compounds is neurotoxicity, which causes peripheral neuropathies including polyneuropathy. It is for this reason, in part, that the development of the method of the present invention is so significant since it now potentially enables treatment protocols to be designed which are either of a shorter duration or which use lower concentrations of platin than previously required. The benefit to patients in terms of reduced side effects is significant Examples of platins include, but are not limited to:
cisplatin (the first to be developed)
carboplatin (a second-generation platinum-based antineoplastic agent)
oxaliplatin
satraplatin
picoplatin
Nedaplatin
Triplatin
Lipoplatin (a liposomal version of cisplatin).

Accordingly, in another embodiment there is provided a method for the treatment of neoplastic condition in a subject, said method comprising:
(i) administering follistatin to said subject for a time and under conditions sufficient for said follistatin to increase the sensitivity of a neoplastic cell to a platin; and
(ii) administering said platin agent to said subject for a time and under conditions sufficient to downregulate said neoplastic cell growth;
wherein said platin is selected from:
   cisplatin
   carboplatin
   oxaliplatin
   satraplatin
   picoplatin
   Nedaplatin
   Triplatin
   Lipoplatin.
   In still another aspect there is provided a method for the treatment of neoplastic condition in a subject, said method comprising:
(i) administering follistatin to said subject for a time and under conditions sufficient for said follistatin to increase the sensitivity of a neoplastic cell to an alkylating agent; and
(ii) administering said alkylating agent to said subject for a time and under conditions sufficient to downregulate said neoplastic cell growth.

In one embodiment, said neoplastic condition is a malignant condition. In another embodiment said malignant condition is a carcinoma or an adenocarcinoma. In yet another embodiment, said neoplastic condition is metastatic.

In still another embodiment, said neoplastic condition is non-small cell lung cancer, breast cancer, pancreatic cancer, mesothelioma, lymphoma, testicular cancer, ovarian cancer, small cell carcinoma, colorectal cancer, oral cancer, head and neck cancer, cervical cancer, bladder cancer or epithelial cancer.

In still another embodiment, said neoplastic condition is characterised by neoplastic cells which exhibit defective DNA repair mechanisms.

In a related aspect it has been determined that simultaneously antagonising both ACVR1B and TGFBR1 produces a level of therapeutic efficacy which is better than antagonising either one of the relevant signalling pathways in isolation.

Accordingly, in a related aspect there is provided a method for the treatment of a neoplastic condition in a subject, said method comprising:
(i) administering to said subject an antagonist of the functionality of ACVR1B and TGFBR1 for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

In one embodiment, said neoplastic condition is a malignant neoplastic condition, in particular a carcinoma.

In another embodiment, said carcinoma is an adenocarcinoma. In yet another embodiment, said carcinoma or adenocarcinoma is of the lung. In still another embodiment, said lung adenocarcinoma is non-small cell lung cancer.

In still yet another embodiment, said neoplastic condition is characterised by neoplastic cells which exhibit defective DNA repair mechanisms.

According to this embodiment there is provided a method for the treatment of a neoplastic condition in a subject, which neoplastic condition is characterised by neoplastic cells that exhibit defective DNA repair mechanisms, said method comprising:
(i) administering to said subject an antagonist of the functionality of ACVR1B and TGFBR1 for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate the neoplastic cell growth.

In another embodiment, said neoplastic condition is malignant, in particular metastatic. In yet another embodiment said neoplastic condition is a carcinoma or an adenocarcinoma.

In still another embodiment, said neoplastic condition is non-small cell lung cancer, breast cancer, pancreatic cancer, mesothelioma, lymphoma, testicular cancer, ovarian cancer, small cell carcinoma, colorectal cancer, oral cancer, head and neck cancer, cervical cancer, bladder cancer or epithelial cancer.

In yet still another embodiment, said neoplastic condition is lung cancer, testicular cancer, ovarian cancer, gastric cancer or bladder cancer.

It should also be understood that where the antagonist to ACVR1B, TGFBR1, GDF11 and Activin A takes the form of multiple antagonistic molecules (as opposed to follistatin which is a single molecule which unexpectedly downregulates the functionality of all these molecules), those antagonists can be administered either simultaneously or in a staggered protocol, such as sequentially. To the extent that they are not administered simultaneously, it would be appreciated that they must nevertheless be administered such that they are able to function to antagonise both ACVR1B and TGFBR1 functionality at the same time, thereby enabling the improved chemosensitivity to be achieved. It should also be understood that the subject antagonist may antagonise the receptor directly, such as an antibody directed to the receptor or siRNA molecules directed to the siRNA encoding the receptors, or it may be directed to preventing a ligand of the receptor from interacting with the receptor, such as an antibody which binds to the ligand and sterically hinders its interaction with the receptor, or a soluble receptor which competitively inhibits ligand binding.

In one embodiment, said antagonists are follistatin, antibodies to the ACVR1B, TGFBR1, GDF11 or Activin A receptors or ligands, siRNA directed to the ACVR1B, TGFBR1, GDF11 and Activin A encoding siRNA, soluble ACVR1B or TGFBR1 receptors, soluble receptors of ACVR1B and TGFBR1 ligands or GDF11 or Activin A.

Yet another aspect of the present invention is directed to the use of:
(i) an antagonist of ACVR1B-mediated signalling; and
(ii) an alkylating agent or a platin agent

in the manufacture of a medicament for the treatment of a neoplastic condition in a subject wherein:

(i) administering said antagonist to said subject downregulates ACVR1B-mediated signalling and increases the sensitivity of a neoplastic cell to an agent which downregulates neoplastic cell growth, which agent is a platin agent or alkylating agent; and
(ii) administering said agent to said subject for a time and under conditions sufficient to downregulates said neoplastic cell growth.

In one embodiment, both ACVR1B and TGFBR1-mediated signalling are downregulated.

Yet another aspect of the present invention is directed to the use of:
(i) an antagonist of GDF11 and activin A; and
(ii) an alkylating agent or a platin agent

in the manufacture of a medicament for the treatment of a neoplastic condition in a subject wherein:
(i) administering said antagonist to said subject increases the sensitivity of a neoplastic cell to an agent which downregulates neoplastic cell growth, which agent is a platin agent or an alkylating agent; and
(ii) administering said agent to said subject for a time and under conditions sufficient to downregulates said neoplastic cell growth.

In one embodiment, said neoplastic condition is a malignant condition. In another embodiment said malignant condition is a carcinoma or an adenocarcinoma. In yet another embodiment, said neoplastic condition is metastatic.

In still another embodiment, said neoplastic condition is non-small cell lung cancer, breast cancer, pancreatic cancer, mesothelioma, lymphoma, testicular cancer, ovarian cancer, small cell carcinoma, colorectal cancer, oral cancer, head and neck cancer, cervical cancer, bladder cancer or epithelial cancer.

In still another embodiment, said neoplastic condition is characterised by neoplastic cells which exhibit defective DNA mechanisms.

In yet still another embodiment, both ACVR1B and TGFBR1 are antagonised.

In another embodiment, said antagonist is follistatin.

In still another embodiment, said antagonist is follistatin, an antibody to the ACVR1B, TGFBR1, GDF11 or Activin A receptors or ligands, siRNA directed to the ACVR1B, TGFBR1, GDF11 or Activin A encoding siRNA or soluble ACVR1B or TGFBR1, or soluble receptors of ACVR1B and TGFBR1 ligands or GDF11 or Activin A.

It would be appreciated that the two steps of the present invention are preferably performed sequentially. However, it should also be understood that this method may be modified to incorporate other steps. For example, one may seek to perform a diagnostic/screening step after step (i) in order to assess the effectiveness of the first step. Such screening step may also be applied later in the treatment regime to monitor the effectiveness of the treatment. As detailed hereinbefore, it would also be appreciated that it is well within the skill of the person in the art, and in light of the teaching provided herein, to select and design an administration protocol for the elements herein described including, for example, the antagonist and the platin or alkylating agent treatment method.

Reference to “growth” of a cell or neoplasm should be understood as a reference to the proliferation, differentiation and/or maintenance of viability of the subject cell, while “down-regulating the growth” of a cell or neoplasm is a reference to the process of cellular senescence or to reducing, preventing or inhibiting the proliferation, differentiation and/or maintenance of viability of the subject cell. In a preferred embodiment the subject growth is proliferation and the subject down-regulation is killing In this regard, killing may be achieved either by delivering a fatal hit to the cell or by delivering to the cell a signal which induces the cell to apoptose.

Reference herein to a “subject” should be understood to encompass humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is a human

As detailed hereinbefore, it has been surprisingly determined that antagonising ACVR1B or the combination of GDF11 and Activin A is protective against nephrotoxicity induced by platin or alkylation agent treatment. This is an unexpected and highly significant outcome which has not previously been reported in relation to agents which purport to improve chemosensitivity. Still further, it has also been determined that in addition to protecting against nephrotoxicity in the context of the use of a platin or alkylating agent, the subject antagonism achieve protection against nephrotoxicity induced by chemotherapy agents more generally, regardless of whether those agents are administered in the context of a neoplastic treatment regime or for some other clinical reason.

Accordingly, in a related aspect there is provided a method of reducing nephrotoxicity in a patient undergoing treatment with a chemotherapy agent, said method comprising administering to said patient an antagonist which downregulates ACVR1B-mediated signalling.

In another embodiment, both ACVR1B and TGFBR1-mediated signalling is antagonised.

In another aspect, there is provided a method of reducing nephrotoxicity in a patient undergoing treatment with a chemotherapy agent, said method comprising administering to said patient an antagonist of the functionality of GDF11 and Activin A.

It should be understood that all of the definitions and embodiments hereinbefore described apply equally to these aspects and embodiments of the present invention.

Reference to a “chemotherapy” agent should be understood as a reference to any agent which is capable of inhibiting the proliferation of rapidly dividing cells, including inducing cell death. Without limiting the present invention in any way, such agents are typically used to treat neoplastic conditions. Examples of chemotherapy agents include, but are not limited to, Actinomycin D, Arsenic Trioxide, Asparaginase, Bleomycin, Busulfan, Carboplatin, Carmustine, Chlorambucil, Cisplatin, Corticosteroids, Cyclophosphamide, Daunorubicin, Docetaxel, Doxorubicin, Epirubicin, Etoposide, Fludarabine, Fluorouracil, Gemcitabine, Hydroxyurea, Idarubicin, Ifosfamide, Irinotecan, Lomustine, Melphalan, Mercaptopurine, Methotrexate, Mitomycin, Mitoxantrone, Oxaliplatin, Paclitaxel, Pemetrexed, Procarbizine, Raltitrexed, Streptozocin, Thioguanine, Thiotepa, Topotecan, Treosulfan, Vinblastine, Vincristine, Vindesine, Vinorelbine.

In one embodiment there is provided a method of reducing nephrotoxicity in a patient undergoing treatment with an alkylating agent or a platin agent, said method comprising administering to said patient an antagonist which downregulates ACVR1B-mediated signalling.

In another embodiment, both ACVR1B and TGFBR1-mediated signalling is antagonised.

In still another embodiment, there is provided a method of reducing nephrotoxicity in a patient undergoing treatment with an alkylating agent or a platin agent, said method comprising administering to said patient an antagonist of the functionality of GDF11 and Activin A.

It would be appreciated by the skilled person that although treatment with a chemotherapy agent is most commonly performed in the context of treatment for a neoplastic condition, it is not inconceivable that treatment with these agents may be applied for other reasons which are not related to cancer. In these situations, the use of an antagonist as hereinbefore defined, such as follistatin, provides a means of reducing the degree and extent of damage to kidney functionality that would otherwise occur. In terms of the applications of this method to neoplastic conditions, this aspect of the present method enables the administration of higher doses of the chemotherapy agent, such as a platin agent, since the kidneys are protected. Still further, some patients are particularly susceptible to kidney damage, such as the elderly or people with diabetes. Accordingly, the method of the present invention provides a means of enabling chemotherapy to be performed such that kidney damage is minimised.

In one embodiment, said patient is undergoing treatment for a neoplastic condition.

In another embodiment, said neoplastic condition is a solid tumour.

In this regard, reference to the therapy of this aspect of the invention should be understood as a reference to the administration of the subject antagonist being an additional step to the treatment with the chemotherapy agent, such as an alkylating or platin agent, and not necessarily being an essential part of that treatment regime. In this regard, any suitable co-administration protocol may be applied, this having been described earlier. For example, one may administer the agents simultaneously or sequentially in one or multiple doses. Still further, it should be understood that the administration of the antagonist need not necessarily proceed entirely in parallel with the chemotherapy agent. For example, the administration of the antagonist may be commenced either before or after the commencement of the chemotherapy agent treatment regime. It may also be concluded either before or after the conclusion of the chemotherapy agent treatment regime. A decision as to how to best to administer the antagonist is well within the skill of the person in the art.

It should also be understood that consistently with the definition of “treatment”, the subject therapy need not completely prevent nephrotoxicity. Rather, it may simply reduce or otherwise ameliorate the nature and extent of the subject toxicity relative to what would otherwise occur if the therapy was not administered. In this regard, reference to “nephrotoxicity” should be understood as a reference to the adverse impact on any one or more aspects of renal functionality which is either directly or indirectly induced by a chemotherapy agent to a patient.

In yet another aspect there is provided the use of an antagonist which downregulates ACVR1B-mediated signalling in the manufacture of a medicament for a condition characterised by treatment with a chemotherapy agent, wherein nephrotoxicity is reduced.

In one embodiment, both ACVR1B and TGFBR1-mediated signalling is antagonised.

In another aspect, there is provided the use of an antagonist of GDF11 and Activin A functionality in the manufacture of a medicament for a condition characterised by treatment with a chemotherapy agent, wherein nephrotoxicity is reduced.

In accordance with these aspects, said chemotherapy agent is preferably an alkylating agent or a platin agent.

It should be understood that the term “treatment” does not necessarily imply that a subject is treated until total recovery. Accordingly, treatment includes reducing the severity of an existing condition, amelioration of the symptoms of a particular condition or preventing or otherwise reducing the risk of developing a particular condition.

Administration of the antagonist and the treatment agent, in the form of pharmaceutical compositions, may be performed by any convenient means. The pharmaceutical composition is contemplated to exhibit therapeutic activity when administered in an amount which depends on the particular case. The variation depends, for example, on the human or animal and the particular agent, ligand and effector mechanism selected for use. A broad range of doses may be applicable. Dosage regimes may be adjusted to provide the optimum therapeutic response.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject alkylating agent or platin may be administered together with an agonistic agent in order to enhance its effects. By “sequential” administration is meant a time difference of from seconds, minutes, hours or days between the administration of the two types of molecules. These molecules may be administered in any order.

In yet another further aspect, the present invention contemplates a pharmaceutical composition comprising the agents as hereinbefore defined together with one or more pharmaceutically acceptable carriers and/or diluents. Said agents are referred to as the active ingredients.

The pharmaceutical forms are preferably suitable for injectable use and include sterile aqueous solutions (where water soluble) or dispersions and sterile powders for the extemporaneous preparation of sterile injectable solutions or dispersion. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. It would be appreciated, for example, that some chemotherapy agents can be delivered orally. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Yet another aspect of the present invention is directed to a kit comprising said agent.

The present invention is further described by reference to the following non-limiting examples.

### EXAMPLE 1

### Modelling Innate Platinum Resistance In Vitro

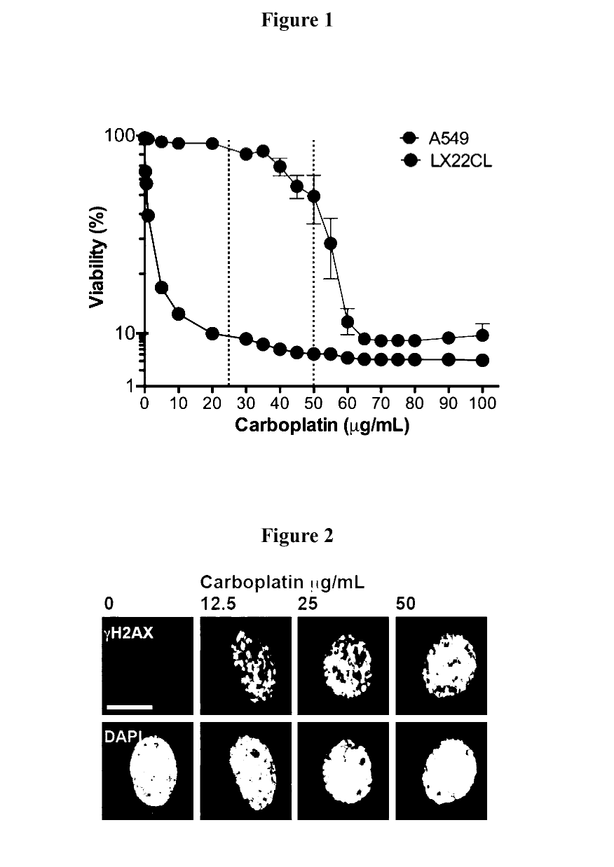

The A549 lung adenocarcinoma cell line was originally derived from a smoker prior to treatment with chemotherapy. This chemonaive cell line carrying a common KRAS mutation was chosen as the primary cell line for modelling innate platinum responsiveness. Platinum responsiveness of A549 lung adenocarcinoma cell line was assessed against a wide range of carboplatin concentrations. FIG. 1 shows typical innate platinum responsiveness of the A549 lung adenocarcinoma cell line compared with the carboplatin response in LX22CL cells, a well characterized small cell lung cancer (SCLC) line from a chemonaive patient. According to the method described by Stewart et. al (2007), FIG. 1 shows that the chemonaive A549 lung adenocarcinoma cell line exhibits an active resistance mechanism, whereas the LX22CL cell line shows sensitivity to carboplatin.

FIG. 2 shows that carboplatin is able to enter A549 lung adenocarcinoma cells and effect DNA damage, at much lower concentrations than is required to induce cell death (compare with FIG. 1). This is consistent with defective DNA repair as an active mechanism of platinum resistance.

### EXAMPLE 2

### Synthetic Lethal Screen to Identify Genes Important for Innate Platinum Resistance

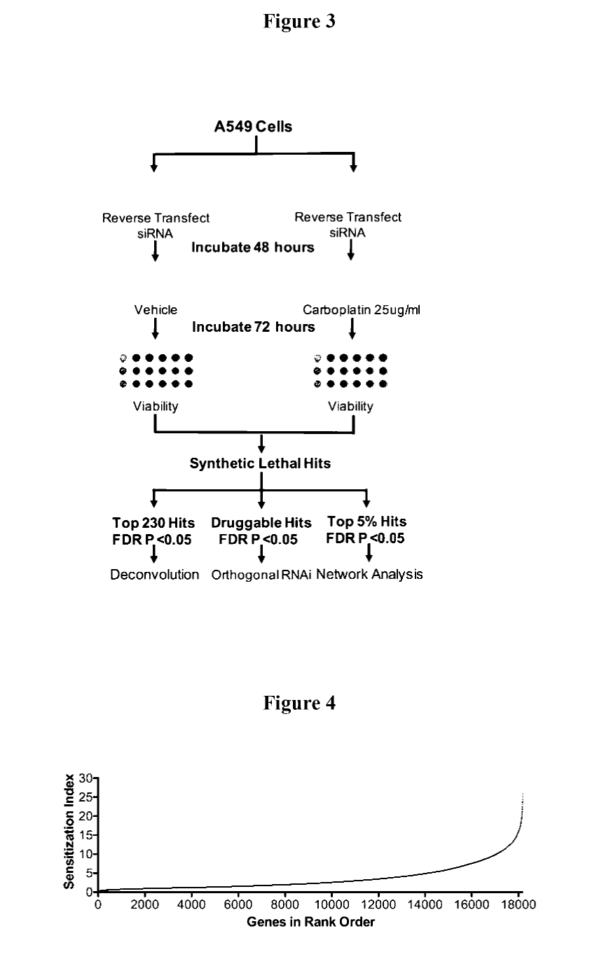

A whole-genome, synthetic-lethal high throughput siRNA screen was completed for platinum-resistance genes using the innately resistant, chemonaive, KRAS mutant A549 lung adenocarcinoma cell line (FIG. 3). Genes were ranked in order of their sensitization index (FIG. 4), which is determined by the fold change difference between the vehicle and sub-lethal dose of platinum. This selects for genes, which in the presence of vehicle have no effect but show a large change in cell viability after addition of the platinum.

From screening of the “druggable” siGenome library 30 candidate therapeutic targets were identified, 14 of which are already in clinical use (Table 1). This analysis identified both ACVR1B and TGFBR1 as potential therapeutic targets for sensitization to platinum.

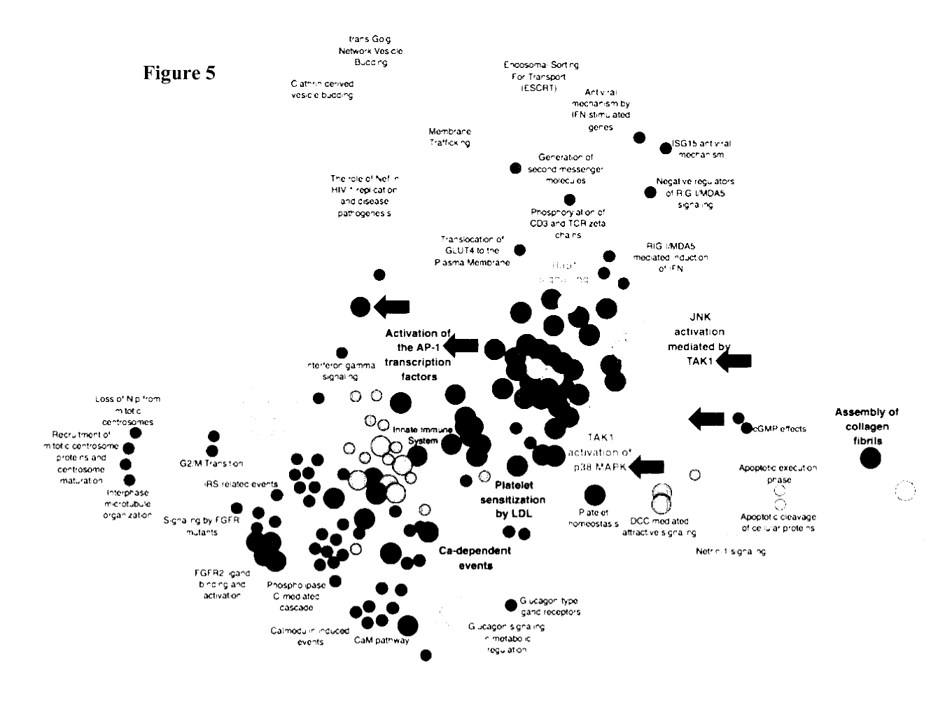

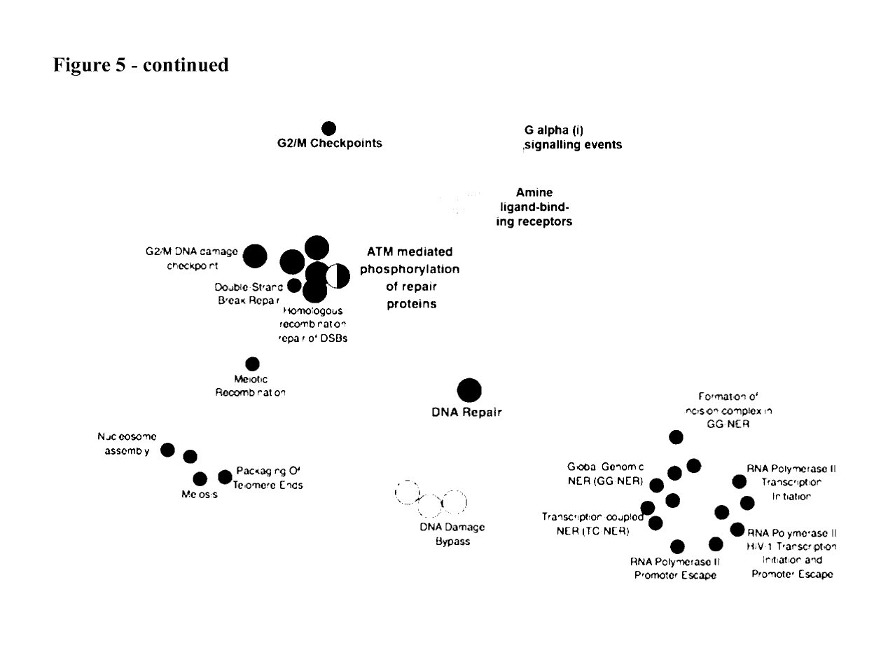

From the top 5% of genes identified as part of the screen, a pathway network was generated (FIG. 5). This analysis categorizes all genes into their respective pathways and does an enrichment analysis to look for over-represented pathways. Nodes are the enriched pathways and edges represent interactions between pathways. The pathway analysis shows a significant enrichment of immune signalling, acting through the JNK, p38, MAPK pathway, which is mediated by TAK1 (TGFβ-activated kinase 1). DNA repair is strongly represented in this analysis and it was hypothesized that the TGFβ super family is signalling through the TAK1, leading to p53 independent DNA repair through p38 and JNK.

### EXAMPLE 3

### In Vitro Signalling Pathway Data

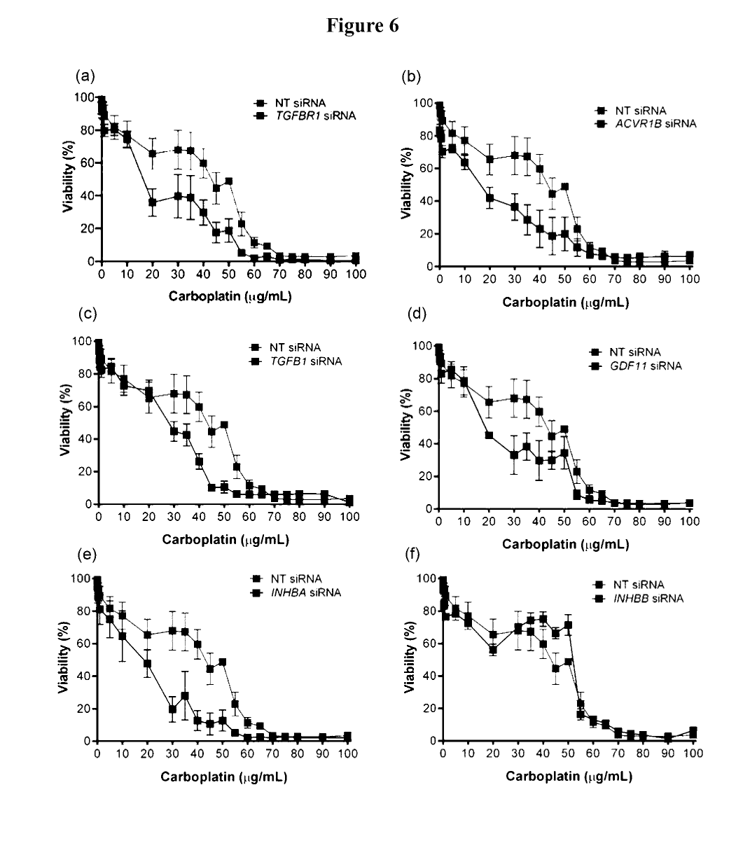

To confirm the results of the screen, orthogonal siRNA validation for both ACVR1B and TGFBR1 as well as ligands that signal through these receptors was performed. Using siRNA independent from the screen in terms of both sequence and chemistry it was found that knockdown of either ACVR1B or TGFBR1 showed sensitization to platinum in A549 lung adenocarcinoma cells (FIG. 6). Knockdown of TGFβ1, which signals through TGFBR1, showed sensitization to platinum in A549 lung adenocarcinoma cells (FIG. 6). The ligands, Activin A (encoded by INHBA gene) and Activin B (encoded by INHBB gene), signal through ACVR1B. However, only Activin A was able to induce sensitization to platinum following siRNA mediated knockdown (FIG. 6). Knockdown of GDF11, a ligand that can signal through both TGFBR1 and ACVR1B receptors, also showed sensitization to platinum (FIG. 6).

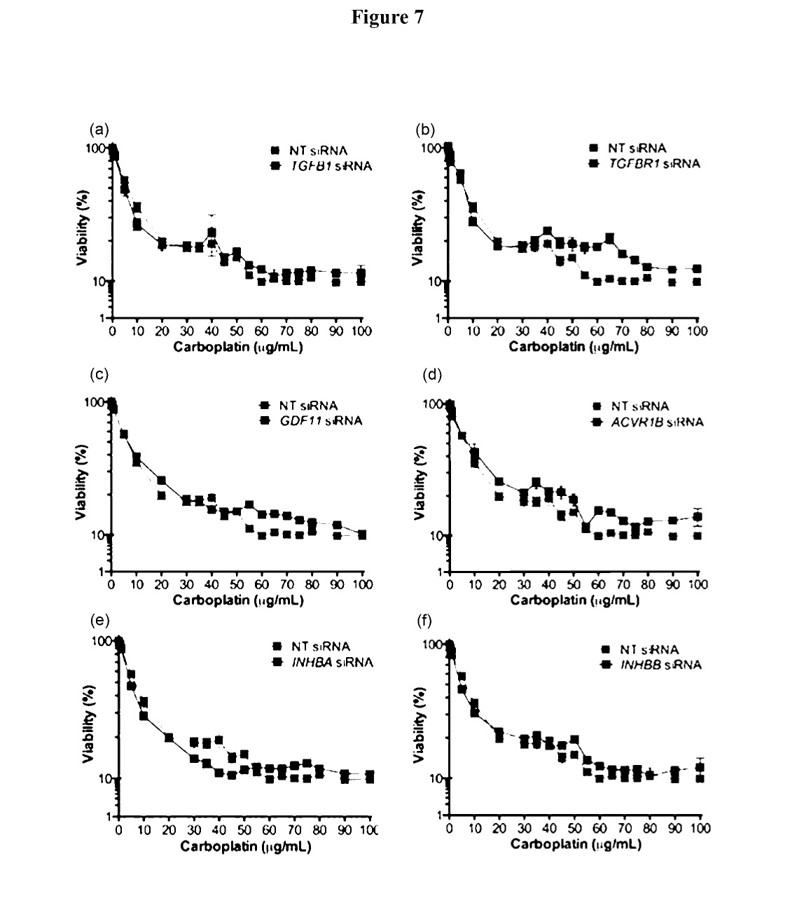

Seeing that siRNA knockdown of ACVR1B and TGFBR1 induced toxicity of neoplastic cells to platinum, it was investigated that whether the same effect will occur in non-neoplastic cells. When tested in a non-neoplastic human kidney cell line, HEK293, no sensitization to platinum was observed after knockdown of ACVR1B or TGFBR1 receptors or the ligands that signal through these receptors (FIG. 7). Instead these kidney cells seem to survive better especially at higher platinum concentrations, suggesting that perhaps knockdown of ACVR1B and TGFB1 is protective to non-neoplastic cells (FIG. 7).

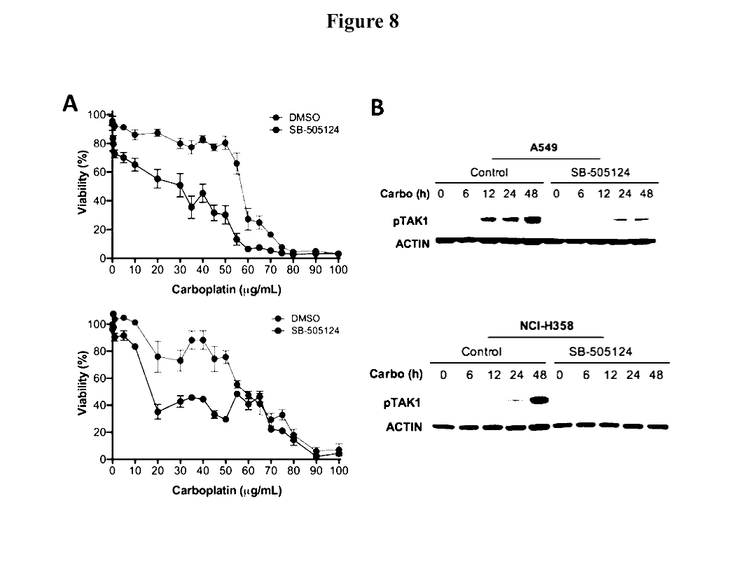

To further validate the result of the screen, the small molecule inhibitor SB-505124, which inhibits the functions of both ACVR1B and TGFBR1 receptors, was used. Inhibition of the function of both ACVR1B and TGFBR1 receptors in the chemoresistant A549 lung adenocarcinoma cells induced sensitization to platinum (FIG. 8A top panel). Given that A549 lung adenocarcinoma cell lines showed sensitization to platinum when treated with SB-505124, the ability of SB-505124 to induced platinum sensitization was tested in another chemonaive and chemoresistant cell line, NCI-H358. This cell line, derived from a patient prior to chemotherapy, also showed sensitization to platinum when treated with the small molecule inhibitor SB-505124 (FIG. 8A bottom panel) further confirming the importance of ACVR1B and TGFBR1 signalling for innate platinum resistance.

Synthetic lethal screen and pathway network analysis identified that TGFβ activated Kinase 1 (TAK1) as a downstream effector for signal transduction for platinum resistance. In cells treated with carboplatin, phosphorylated TAK1 (activated form) levels were upregulated (FIG. 8B). Treatment of cells with SB-505124 inhibited the activation of TAK1 and sensitized platinum-resistant A549 and NCI-H358 cells to carboplatin in vitro (FIGS. 8B), suggesting that TAK1 activation via ACVR1B/TGFBR1 signalling is required for platinum resistance.

### EXAMPLE 4

### Use of Other Alkylating Agents to Model Innate Chemoresistance Sensitization

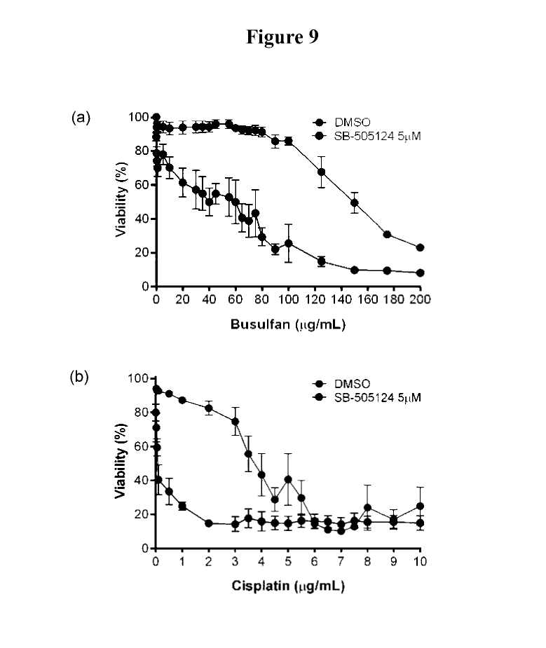

The therapeutic potential of ACVR1B and TGFBR1 signalling to regulate innate resistance of adenocarcinomas was investigated using other alkylating agents in addition to carboplatin. Inhibition of ACVR1B/TGFBR1 in the presence of cisplatin or busulfan, two members of the classical alkylating agents, showed sensitization in A549 lung adenocarcinoma cells (FIG. 9). This shows that inhibition of ACVR1B/TGFBR1 can be used in conjunction with a wide range of alkylating agents to sensitize the innate resistance of neoplastic cells.

### EXAMPLE 5

### Modelling Innate Platinum Resistance In Vivo

Follistatin (FST) is a molecule that is known for its role in regulating the activities of TGFβ superfamily members, including activin and GDF11, both of which were identified as important players in chemoresistance of non-small cell lung carcinoma (NSCLC) cells (see Example 2). Therefore, the effects of FST on inducing sensitization of NSCLC cells to platinum therapy were assessed in an in vivo xenograft model. Athymic nude mice bearing A549 flank xenografts were treated with combinations of vehicle control, 60 mg/kg carboplatin on day 0, and/or 2 μg FST on day −1, +1, +3 and +5. Five mice were randomized in to each of the treatment groups and treatments started when the tumour size was 200 mm³. Tumour volumes were measured daily and the animals were culled when the tumour size reached over 500 mm³.

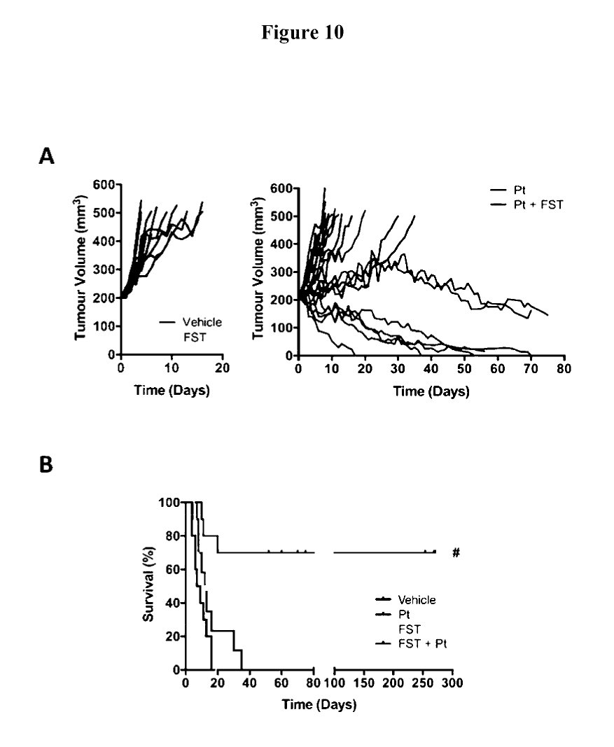

FIG. 10 shows that administration of platinum or FST alone did not affect tumour growth, so that tumours grew at the same rate as that seen in control (untreated) mice and all mice were culled within 20 days. However, when FST was administered with platinum therapy, tumour growth was significantly decreased, with 7 out of the 10 animals displaying a regression in tumour volume and continuing to be tumour free for up to 271 days. This data shows that FST induces sensitivity of the tumour to the platinum therapy thereby increasing the effectiveness and improving the success of treating cancers with chemotherapy agents; cancers that would otherwise have not responded to this treatment.

### EXAMPLE 6

### Modelling Nephrotoxicity of Platinum Treatment In Vivo

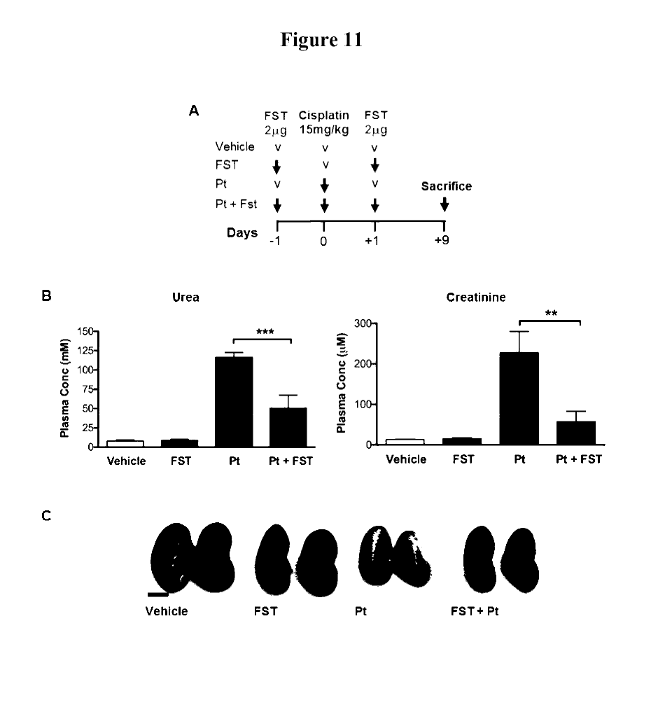

The siRNA treatment showed that targeting ACVR1B, TGFBR1, INHBA or GDF11 did not increase the toxicity of platinum to HEK293 cells. Therefore, the effect of FST on platinum induced kidney toxicity was investigated in vivo. Adult mice were treated with combinations of vehicle control, 5 mg/kg cisplatin on day 0, and/or 2 μg FST on day −1 and +1 (FIG. 11A). Five mice were randomized into each of the treatment groups. Animals were culled on day +9 and macroscopic kidney morphology and plasma levels of creatinine and urea were analyzed.

FIG. 11B shows that administration of vehicle or FST alone did not affect the plasma concentrations of either urea or creatinine. However, platinum treatment dramatically elevated the plasma levels of urea and creatinine (FIG. 11B), indicative of kidney damage, and resulted in small and pale kidneys (FIG. 11C) consistent with platinum induced cell death and ischemia. Administration of FST in conjunction with platinum therapy, significantly attenuated the cisplatin-induced nephrotoxicity as evidenced by overall kidney morphology as well as plasma concentrations of urea (P<0.001) and creatinine (P<0.01) (FIG. 11B and 11C). This data shows that FST protects the kidneys from platinum induced toxicity.

### EXAMPLE 7

### Clinical Significance

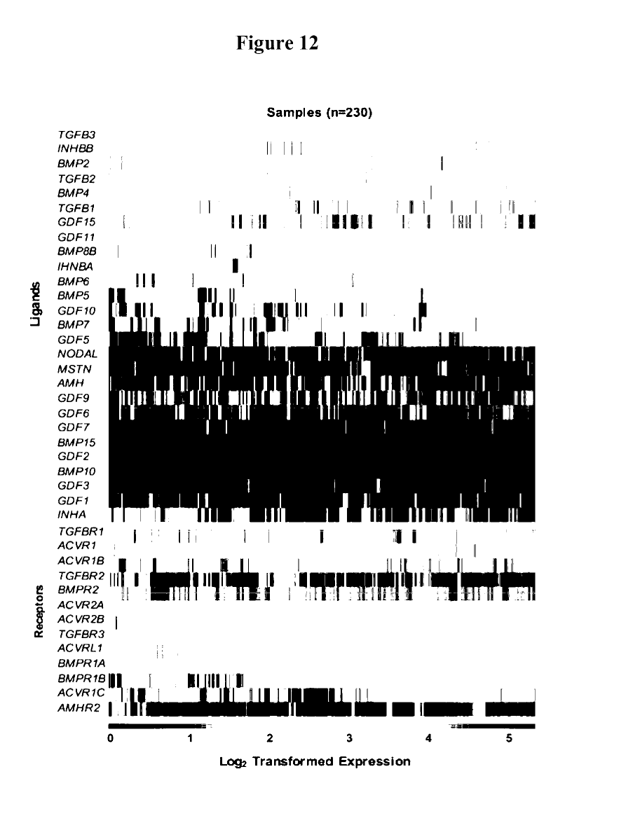

To confirm that the targets identified from the screen were viable as therapeutic targets for use in treating lung adenocarcinomas their expression levels were investigated across a panel of 230 cases of lung adenocarcinoma samples, comprising the TCGA Lung Adenocarcinoma study (FIG. 12). From this analysis it was found that among number of highly upregulated receptors both TGFBR1 and ACVR1B are expressed at a level beyond that of TTF1, a known marker used to identify adenocarcinoma clinically. Of the ligands, TGFB1 and INHBB upregulation was over represented in TCGA lung adenocarcinoma study compared to upregulation of GDF11 and INHBA. However, despite this significant upregulation, INHBB was found to have no effect on platinum sensitization (see Example 3). This suggests that the observed differential gene expression is not directly indicative of genes that confer platinum resistance.

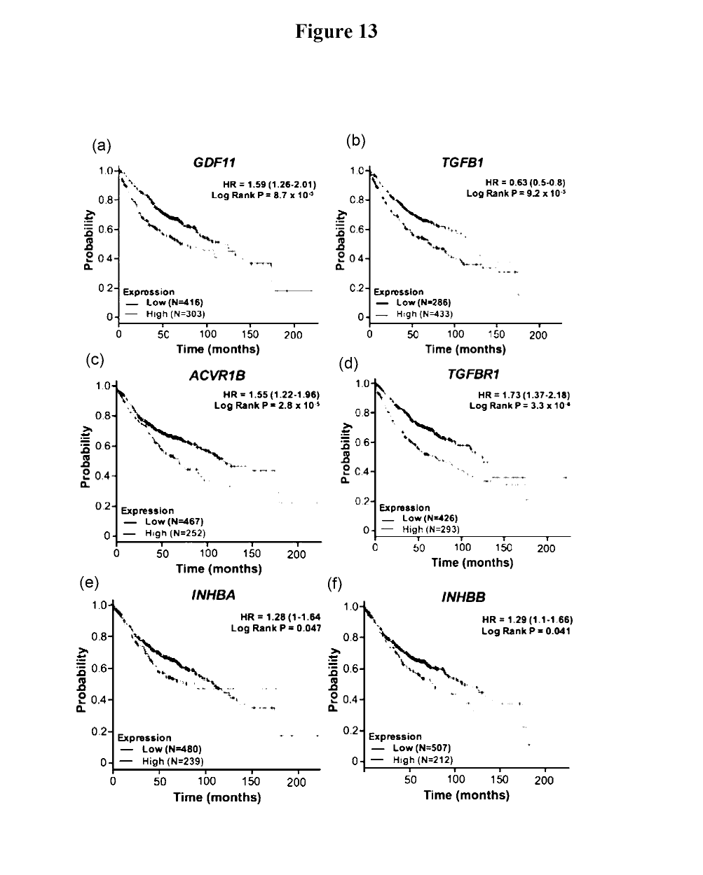

Further analysis of the associations between the expression of TGFβ superfamily genes in lung adenocarcinoma and overall survival showed that higher expression levels of TGFBR1 and ACVR1B correlated with lower survival probability (FIG. 13). In stark contrast, over expression of TGFB1 is a good prognostic marker for overall survival, suggesting that TGFB1 is much less likely to drive chemoresistance than Activin A or GDF11. In conjunction with the results on platinum sensitization, this suggests that Activin A, GDF11 and ACVR1B are viable therapeutic targets in treating adenocarcinoma.

### EXAMPLE 8

### Materials and Methods for Examples 1-7

Assessing the innate levels of chemoresistance in lung cancer: 10,000 cells per well were plated in to a 96 well plate. After cells had attached to the plate media was removed and replaced with increasing concentrations of alkylating agent, carboplatin, cisplatin or busulfan, in quadruplicate with appropriate controls. Plates are left to incubate for 3 days with an Alamar Blue reading performed at end point.

Curves of cell death in response to the respective alkylating agent were generated by graphing fluorescence (as a measure of cell viability) vs the alkylating agent concentration. From this data set, conventional comparative parameters such as IC₅₀ can be extracted and used to determine the effect of carboplatin, cisplatin or busulfan on each cell line.

DNA damage induced by platinum was visualised by confocal immunofluorescence microscopy. A549 lung adenocarcinoma cells were treated with appropriate primary and secondary antibodies to visualise γH2AX and the DNA counterstain DAPI following 24 hr treatment with increasing concentrations of carboplatin.

Whole Genome Synthetic Lethal Screen: 10 μL of siRNA, OptiMEM and Lipofectamine RNAi Max mix was plated in to a 384 well plate. 30 μL of cells were plated on to this solution to give a final volume of 40 μL and a final siRNA concentration of 40 nM and 2000 cells per well. Plate was left to incubate overnight and next day media was removed from the plate and replaced with 30 μL of fresh media. The next day 10 μL of media contained either vehicle or platinum was added (final platinum concentration25 μg/mL). After 5 days of incubation 4 μL of Alamar Blue was added and plates were incubated for 5 hours before being read using Fluostar Optima Plate Reader.

Cytoscape Pathway Analysis: Gene ontology and pathway network analysis were performed using the ClueGo application of the open-source Cytoscape platform. ClueGo generates pathway term networks by performing enrichment analysis on a gene list of interest, whilst utilizing the Reactome pathway database as a frame of reference.

Sensitization of Cells to platinum with siRNA: Non-targeting, or TGFBR1, ACVR1B, TGFB1, GDF11, INHBA or INHBB siRNA was reverse transfected into cells. Here, 5000 cells per well were plated in the siRNA/lipofectamine RNAi Max solution and allowed to adhere. After two days, increasing concentrations of platinum were added to wells in quadruplicate. Plates were left to incubate for 5 days, with Alamar Blue reading performed on day 3 and 5 post platinum.

Sensitization of Cells to platinum with TGFBR1 and ACVR1B inhibitor: 5000 cells per well were plated in quadruplicates in RPMI with 1% heat-inactivated NCS (HiNCS) and Glutamax and allowed to adhere overnight. Media was then removed and either vehicle or 5 μM SB-505124 was added and incubated for 24 hours. Media was then removed and replaced with media containing increasing concentrations of carboplatin with either vehicle or 5 μM SB-505124. After 3 days of incubation Alamar Blue was added and plates were incubated for 2.5 hours before being read using Fluostar Optima Plate Reader.

Western blots: Cell lysates were prepared using RIPA buffer with the addition of Halt Protease and Phosphatase Inhibitor Cocktails. Gel electrophoresis and transfer was performed using the BioRad MiniProtean System according to manufacturer's instructions. Phospho-TAK1 was detected using the combination of Rabbit anti-Phospho-TAK1 and anti-Rabbit IgG, HRP linked primary and secondary antibodies respectively.

in vivo modelling of platinum resistance: Athymic nude mice bearing A549 flank xenografts were treated with combinations of vehicle control, 60 mg/kg carboplatin on day 0, and/or 2 μg FST on day −1, +1, +3 and +5 administered intraperitoneally. Five mice were randomized in to each of the treatment groups and treatments started when the tumour size was 200 mm³. Tumour volumes were measured daily and the animals were culled when the tumour size reached over 500 mm³. Recombinant human Follistatin 288 (FST) was used in all experiments. FST was provided by Paranta Biosciences Ltd.

in vivo modelling of platinum induced nephrotoxicity: Adult mice were treated with combinations of vehicle control, 5 mg/kg cisplatin on day 0, and/or 2 μg FST on day −1 and +1 administered intraperitoneally. Five mice were randomized in to each of the treatment groups. Animals were culled on day +9 and macroscopic kidney morphology and plasma levels of creatinine and urea were analysed. Recombinant human FST 288 (FST) was used in all experiments. FST was provided by Paranta Biosciences Ltd.

TCGA Analysis: The human gene expression data was downloaded from the TCGA lung adenocarcinoma study through cBioPortal (Gao et al 2013, Cerami et al. 2012), in which RNA of 230 tumours was sequenced by Illumina HiSeq, aligned by Mapsplice, and quantified by RSEM. Heatmap: Heat maps were generated using heatmap.2 function within the gplots R/Bioconductor package. Gene expression levels of human tumours were log₁₀ transformed in the heatmap.

TABLE 1
High Confidence Hits Targetable with Existing Small or Large Molecules.

| | | FDR P | | | |
|---|---|---|---|---|---|
| Gene | SI | value | Description | Inhibitor | Phase |
| ABCC6 | 10.06 | 0.0017 | ATP-binding cassette, | MK571, Probenecid, | 0 |
| | | | sub-family C | Indomethacin | |
| | | | (CFTR/MRP), member 6 | | |
| ACVR1B | 16.35 | 5.838 × 10⁻⁶ | Activin receptor 1B | SB-431542, SB-505124 | 0 |
| ADCY1 | 17.62 | 5.059 × 10⁻⁵ | Adenylate cyclase 1 | SQ-22536 | 0 |
| AHR | 11.47 | 0.00041 | Aryl Hydrocarbon | CH-223191, DiMNF | 0 |
| | | | Receptor | | |
| AKR1C1 | 12.48 | 1.536 × 10⁻⁵ | Aldo-keto reductase | Medroxyprogesterone, | 4 |
| | | | family 1, member C1 | acetylsalicylic acid, | |
| | | | | diazepam | |
| ASAH3 | 9.48 | 0.00012 | Alkaline ceramidase 1 | Ceranib 1 | 0 |
| BCL2L1 | 13.8 | 0.0014 | BCL2 family anti- | ABT-737, ABT-263 | 3 |
| | | | apoptosis protein | | |
| | | | (BCLXL) | | |
| CA5B | 9.75 | 0.0048 | Carbonic anhydrase VB, | Zonisamide, Ellagic acid | 4 |
| | | | mitochondrial | | |
| CAMKK2 | 17.32 | 1.518 × 10⁻⁵ | Ca(2+)/calmodulin- | STO-609, A-484954, KN- | 0 |
| | | | dependent | 93, Arcyriaflavin A | |
| | | | serine/threonine kinase | | |
| CSNK1A1 | 15.9 | 0.0019 | Casein kinase 1, alpha 1 | D-4476, (R)-DRF053 | 0 |
| CYP1A2 | 9.73 | 0.0006 | Cytochrome P450, family | Fluvoamine, Clozapine, | 4 |
| | | | 1, subfamily A, | Erlotinib, Melatonin, | |
| | | | polypeptide 2 | Amitryptiline | |
| CYP2A13 | 18.07 | 0.0002 | Cytochrome P450, family | Methoxalen | 4 |
| | | | 2, subfamily A, | | |
| | | | polypeptide 13 | | |
| FKBP3 | 9.77 | 1.320 × 10⁻⁵ | FK506 binding protein 3, | Tacrolimus, Rapamycin | 3 |
| | | | 25 kDa | | |
| HDAC3 | 9.48 | 0.0002 | Histone deacetylase 3 | Tacedinaline, Entinostat, | 3 |
| | | | | LBH589 | |
| HSPA4 | 10.06 | 0.0004 | Heat shock protein, | Geldanamycin | 2 |
| | | | 70 kDa, protein A4 | | |
| HSPB8 | 14.83 | 8.869 × 10⁻⁶ | Heat shock protein, | Geldanamycin | 2 |
| | | | 70 kDa, protein B8 | | |
| HSPCA | 9.83 | 0.0003 | Heat shock protein, | 17-AAG | 2 |
| | | | 90 kDa, alpha (cytosolic), | | |
| | | | class A, 1 | | |
| IL12B | 9.41 | 8.813 × 10⁻⁶ | Interleukin 12 beta | Ustekinumab | 3 |
| IL4R | 11.67 | 0.00037 | Interleukin 4 receptor | Dupilumab | 3 |
| MAPK1 | 17.41 | 0.0014 | Mitogen-activated protein | FR-180204 | 0 |
| | | | kinase 1 (p42 MAPK) | | |
| MAPK11 | 14.20 | 0.0002 | Mitogen-activated protein | Doramapimod, | 1 |
| | | | kinase 11 (p38 MAPK | Losmapimod, LY-2228820, | |
| | | | beta) | PD-169316 | |
| MCL1 | 10.25 | 0.0009 | Myeloid cell leukemia 1 | Navitoclax, Obatoclax, | 3 |
| | | | | MIM1 | |
| NOS3 | 9.38 | 0.0139 | Nitric ozide synthase 3 | L-NIO Dihydrochloride | 0 |
| | | | (eNOS) | | |
| NR2C2 | 8.95 | 0.0249 | Nuclear receptor | CBP501 | 0 |
| | | | subfamily 2, group C, | | |
| | | | member 2 (TAK1) | | |
| PARP1 | 15.82 | 8.249 × 10⁻⁵ | Poly (ADP-ribose) | Veliparib | 3 |
| | | | polymerase 1 | | |
| PDE6G | 12.55 | 0.003 | Phosphodiesterase 6G, | IBMX | 0 |
| | | | CGMP-specific, rod, | | |
| | | | gamma | | |
| PNLIP | 9.27 | 9.888 × 10⁻⁵ | Pancreatic lipase | Orlistat, Pristimerin | 0 |
| POLG2 | 10.23 | 0.0002 | Polymerase (DNA | Carbovir, DDC, | 4 |
| | | | directed), gamma 2, | Zidovidine, Lamivudine | |
| | | | accessory subunit 1 | | |
| RAC3 | 11.38 | 0.0022 | Ras-related C3 botulinum | EHT-1864, W-56 | 0 |
| | | | toxin substrate 3 | | |
| SLC1A7 | 11.5 | 0.0015 | Solute carrier family 1, | TFB-TBOA, WAY 213613 | 0 |
| | | | (glutamate transporter), | | |
| | | | member 7 | | |
| SLC6A11 | 8.97 | 0.0419 | Solute carrier family 6 | Tiagabine | 4 |
| | | | (neurotransmitter | | |
| | | | transporter), 11 | | |
| SLC6A4 | 17.87 | 1.046 × 10⁻⁵ | Solute carrier family 6 | Paroxetine, Sertraline, | 4 |
| | | | (neurotransmitter | Citalopram, Fluoxetine | |
| | | | transporter), 41 | | |
| SRD5A2L | 9.13 | 0.0129 | Steroid 5 apha-reductase 3 | Finasteride | 4 |
| TGFBR1 | 15.94 | 0.0001 | Transforming growth | Ly-364947, SB-525334, | 0 |
| | | | factor receptor 1 | SB-431542, SD-208 | |
| TLR8 | 12.7 | 0.0002 | Toll-like receptor 8 | Mianserin | 4 |
| VEGFB | 14.34 | 6.043 × 10⁻⁵ | Vascular endothelial | Bevacizumab | 4 |
| | | | growth factor B | | |

Genes listed in bold have been implicated in mediating platinum resistance.
SI = sensitization index.
FDR = false discovery rate.
Phase = phase of therapeutic use in humans.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bender, H., Takahashi, H., Adachi, K., Belser, P., Liang, S. H., Prewett, M., Schrappe, M., Sutter, A., Rodeck, U. and Herlyn, D. (1992) Immunotherapy of human glioma xenografts with unlabeled, 1311-, or 1251-labeled monoclonal antibody 425 to epidermal growth factor receptor. Cancer Research 52: 121-126
Bernard et al. 2004, Molecule Endocrinol 18:606-623
Britz-Cunningham, S. H. and Adelstein, S. J. (2003). Molecular targeting with radionuclides: state of the science. Journal of Nuclear Medicine 44: 1945-1961
Bunin et al. 1994, Proc Natl Acad Sci USA 91:4708-4712
Jennifer N. Cash, Elizabeth B. Angerman, Henry T. Keutmann, and Thomas B. Thompson 2012, Characterization of Follistatin-Type Domains and Their Contribution to Myostatin and Activin A Antagonism. Mol Endocrinol, 26(7):1167-1178
Cerami, E., J. Gao, U. Dogrusoz, B. E. Gross, S. O. Sumer, B. A. Aksoy, A. Jacobsen, C. J. Byrne, M. L. Heuer, E. Larsson, Y. Antipin, B. Reva, A. P. Goldberg, C. Sander, and N. Schultz (2012). “The cBio Cancer Genomics Portal: An Open Platform for Exploring Multidimensional Cancer Genomics Data”. Cancer Discovery. 2(5): p. 401-4.
Chalk et al. 2004, Biochem Biophys Res Commun 319:264-274
Christiansen, J. and Rajasekaran, A. K. (2004). Biological impediments to monoclonal antibody-based cancer immunotherapy. Molecular Cancer Therapy 3: 1493-1501.
Conrad et al. 1995, Mol Divers 1:69-78
Cui et al. 2004, Comput Methods Programs Biomed 75:67-73
Dadachova, E., Nosanchuk, J. D., Shi, L., Schweitzer, A. D., Frenkel, A., Nosanchuk, J. S. and Casadevall, A. (2004). Dead cells in melanoma tumours provide abundant antigen for targeted delivery of ionizing radiation by a mAb to melanin. Proceedings of the National Academy of Sciences of the United States of America 101: 14865-14870
DeWitt et al. 1993, Proc Natl Acad Sci USA 90:6909-6913
Ding, et al., Nucl Acids Res 32 Web Server issue, W135-W141
Ding & Lawrence 2003, Nucl Acids Res 31:7280-7301
Ding & Lawrence 2001, Nucl Acids Res 20:1034-1046
Douillard and Hoffman 1981, Basic Facts about Hybridomas, in Compendium of Immunology Vol Ill., ed. by Schwartz; Kohler and Milstein 1975, Nature 256:495-499
Dussiot, M., T. T. Maciel, et al. (2014). “An activin receptor IIA ligand trap corrects ineffective erythropoiesis in β-thalassemia.” Nature medicine 20(4): 398-407
Elbashir et al. 2000, Methods 26:199-213
Elbashir et al. 2002, Methods 26:199-213
Ellington 1994, Curr Biol 4:427-429
Gamer, L. W., N. M. Wolfman, et al. (1999). Developmental biology 208: 222-232
Gao, J., B. A. Aksoy, U. Dogrusoz, G. Dresdner, B. Gross, S. O. Sumer, Y. Sun, A. Jacobsen, R. Sinha, E. Larsson, E. Cerami, C. Sander, and N. Schultz (2013). “Integrative Analysis of Complex Cancer Genomics and Clinical Profiles Using the cBioPortal”. Science Signaling. 6(269): p. pl1-pl.
Gray, P. C., C. A. Harrison, et al. (2003). PNAS USA 100: 5193-5198
Griffiths, G. L., Govindan, S. V., Sgouros, G., Ong, G. L., Goldenberg, D. M. and Mattes, M. J. (1999). Cytotoxicity with Auger electron-emitting radionuclides delivered by antibodies. International Journal of Cancer 81: 985-992
Harrison et al. 2004, J Biol Chem 279:28036-28044
Kassis et al., Nucleotide Med 46:4S-12S, 2005
Kelland, L. (2007). “The resurgence of platinum-based cancer chemotherapy”. Nature Reviews Cancer 7 (8): 573-0584. doi:10.1038/nrc3167 (https://dx.doi.org/10.10382Fnrc2167). PMID 17625587 (https://www.ncbi.nlm.nih.gov/pubmed/17625587)
Henry T. Keutmann, Alan L. Schneyer and Yisrael Sidis 2004 The Role of Follistatin Domains in Follistatin Biological Action. Mol Endocrinol, 18(1):228-240
Khvorova et al. 2003, Cell 115:209-216
Klug et al. 1994, Mol Biol Rep 20:97-107
Kohler and Milstein 1975, Nature 256:495-499
Kohler and Milstein 1976, Eur J Immun 6:511-519
Lato et al. 1995, Chem Biol 2:291-303
T S Lawrence, Oncology (Huntington) 17, 23-28, 2003
Liu, S. and Edwards, D. S. (2001). Bifunctional chelators for therapeutic lanthanide radiopharmaceuticals. Bioconjugate Chemistry 12: 7-34
Loehrer P J, Einhorn L H 1984, Drugs five years later. Cisplatin. Ann Intern Med. 100(5):704-13.
Maeshima et al. 2004, Endocrinology 145:3739-3745
Makanji Y et al. 2011 Generation of a specific activin B antagonist by modification of the activin B propeptide. Endocrinol 152:3758-3768
Morrison, B. M., J. L. Lachey, et al. 2009 (Experimental neurology 217: 258-268) Suragani, R. N. V. S., S. M. Cadena, et al. 2014 (Nature medicine 20(4): 408-414)
O'Donoghue, J. A., Bardies, M. and Wheldon, T. E. (1995). Relationships between tumour size and curability for uniformly targeted therapy with beta-emitting radionuclides. Journal of Nuclear Medicine 36: 1902-1909
Pesch et al. Int J Cancer 2012 131:1210-19
Poulaki et al. 2004, Am J Pathol 164:1293-1302
Reynolds et al. 2004, Nat Biotechnol. 22:326-330
Schwarz, et al. 2003, Cell 115:199-208
Sellers, W. R. and Fisher, D. E. (1999). Apoptosis and cancer drug targeting. Journal of Clinical Investigation 104: 1655-1661
Sozzi, (2001) Eur J Cancer; 37 Suppl 7:S63-73
Stein and Cohen, 1988, Cancer Res 48:2659-2668
Takabe et al. 2003, Hepatology 38:1107-1115
Uphoff et al. 1996, Curr Opin Struct Biol 6:281-287
van der Krol et al., 1988, Biotechniques 6:958-976
Waldmann, T. A. (1991). Monoclonal antibodies in diagnosis and therapy. Science 252: 1657-1662
Wallis et al. 1995, Chem Biol 2:543-552
Wang et al. 2004, Bioinformatics 20:1818-1820
Whittemore and Song 2003 (Biochem & Biophys Res. Comm 300(4): 965-971
Xu et al. 1995 J Biol Chem 270:6308-6313 Yang, Z., J. Zhang, et al. 2008 The Journal of Gene Medicine 10(8): 825-833)

1. A method for the treatment of a neoplastic condition in a subject, said method comprising:
(i) administering to said subject an antagonist which downregulates ACVR1B-mediated signalling for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

2. (canceled)

3. A method for the treatment of a neoplastic condition in a subject, said method comprising:
(i) administering to said subject an antagonist of the functionality of each GDF11 and activin A for a time and under conditions sufficient for said antagonist to increase the sensitivity of a neoplastic cell to an agent which downregulates neoplastic cell growth, which agent is an alkylating agent or a platin agent; and
(ii) administering said agent to said subject for a time and under conditions sufficient to downregulate said neoplastic cell growth.

4. (canceled)

5. The method according to claim 1, wherein said neoplastic condition is selected from one or more of:
(i) a carcinoma or an adenocarcinoma; and/or
(ii) a malignant neoplastic condition; and/or
(iii) a metastatic malignant neoplastic condition, and/or
(iv) is characterized by cells which exhibit defective DNA repair mechanisms.

6-8. (canceled)

9. The method according to claim 1, wherein said neoplastic condition is selected from non-small cell lung cancer, breast cancer, pancreatic cancer, mesothelioma, lymphoma, testicular cancer, ovarian cancer, small cell carcinoma, colorectal cancer, oral cancer, head and neck cancer, cervical cancer, bladder cancer, and epithelial cancer.

10. The method according to claim 1, wherein said ACVR1B antagonist is administered together with a TGFBR1 antagonist.

11. The method according to any claim 1, wherein said antagonist is a proteinaceous or non-proteinaceous molecule which antagonizes the transcription or translation of a gene selected from the ACVR1B gene, the TGFBR1 gene, the GDF11 gene, and the activin βA gene.

12. The method according to claim 11, wherein said proteinaceous molecule is an antibody directed to DNA or RNA.

13. The method according to claim 11, wherein said non-proteinaceous molecule is selected from an antisense oligonucleotide, a DNAzyme, aptamer, and a molecule suitable for use in co-suppression.

14. The method according to claim 1, wherein said antagonist is a proteinaceous or non-proteinaceous molecule which antagonizes ACVR1B, TGFBR1, GDF11 or activin A.

15. The method according to claim 14, wherein said antagonist is selected from:
(i) follistatin;
(ii) the α subunit of inhibin;
(iii) inhibin;
(iv) an antibody directed to activin A, the activin βA subunit, GDF11, ACVR1B or TGIβR1;
(v) a non-functional ACVR1B or TGFBR1 mutant;
(vi) a non-functional GDF11 or activin A;
(vii) a soluble receptor of activin A or GDF11, a soluble ACVR1B or TGFβ1;
(viii) a thrombin antagonist;
(ix) the Cripto protein;
(x) Beta-glycan, and BAMBI, and Follistatin-like 3;
small molecule inhibitors of ACVR1B or TGFBR1; and
(xii) soluble receptors or ligand traps.

16. The method according to claim 14, wherein said follistatin is selected from:
(i) wild-type follistatin (FS), comprising an N-terminal domain (ND) followed by three follistatin domains (FSD1, FSD2 and FSD3) with a heparin-binding sequence located in FSD1 (amino acid sequence positions 72-86), and all known isoforms thereof;
(ii) wild-type follistatin-like 3 protein (FSTL3) (also known as follistatin-related gene product (FLRG) and follistatin-related protein (FSRP)), comprising an N-terminal domain (N3D) followed by two follistatin-like 3 domains (FS3D1 and FS3D2), and all known isoforms thereof;
(iii) a follistatin analogue having the structure ND-FSD1-FSD2 (i.e. wild-type minus FSD3;
(iv) analogues of (i) and (iii) above with FSD1 substituted by FSD1′, where FSD1′ represents FSD1 with the heparin-binding site removed;
(v) analogues of (i) and (iii) above with FSD1 substituted by FSD1*, where FSD1* represents FSD1 with the sequence prior to and including the heparin-binding sequence removed;
(vi) hybrid forms of (i) and (iii) above where at least one of the domains is substituted by a corresponding FSTL3 domain N3D, FS3D1 and FS3D2;
(vii) hybrid forms of (ii) above where at least one of the domains is substituted by a corresponding FS domain ND, FSD1, FSD1′, FSD1* and FSD2;
(viii) any of the above proteins modified by one or more deletions, insertions and/or mutations in ND, N3D, FSD1, FSD1′, FSD1*, FS3D1, FSD2, FS3D2, and FSD3, provided the modified protein functions as an activin B antagonist; and
(ix) genetically modified forms of follistatin which have been modified to preferentially antagonize activin B over other activin or follistatin targets.

17. The method according to claim 1, wherein said alkylating agent is a classical alkylating agent.

18. The method according to claim 17, wherein said classical alkylating agent is selected from a nitrogen mustard, nitrosaureas, and alkyl sulfonate.

19. The method according to claim 18, wherein:
(i) said nitrogen mustard is selected from cyclophosphamide; mechlorethamine or mustine (HN2); uramustine or uracil mustard; melphalan; chlorambucil; Ifosfamide; and bendamustine;
(ii) said nitrosoureas is selected from carmustine; lomustine; and streptozocin; and
(iii) said alkyl sulfonate is busulfan.

20. The method according to claim 1, wherein said alkylating agent is a non-classical alkylating agent.

21. The method according to claim 20, wherein said non-classical alkylating agent is selected from procarbazine; altretamine; and dicarbazine, mitozolomide and temozolomide.

22. The method according to claim 1, wherein said platin is selected from cisplatin; carboplatin; oxaliplatin; satraplatin; picoplatin; Nedaplatin; Triplatin; and Lipoplatin.

23. A method of reducing nephrotoxicity in a patient undergoing treatment with a chemotherapy agent, said method comprising administering to said patient an antagonist which downregulates ACVR1B-mediated signalling.

24. A method of reducing nephrotoxicity in a patient undergoing treatment with an alkylating agent or a platin agent, said method comprising administering to said patient an antagonist of the functionality of GDF11 and Activin A.

25-40. (canceled)

41. The method according to claim 1, wherein said mammal is a human.


SEQUENCE LISTING (2 sequences)

SEQ ID NO: 1 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 2 - protein, 109 aa, Homo sapiens
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS